(19) United States
(12) Patent Application Publication
(10) Pub. No.: US 2005/0148048 A1
(43) Pub. Date: Jul. 7, 2005

(54) Process for producing dipeptides

(75) Inventors: Shin-ichi Hashimoto, Hofu-shi (JP); Kazuhiko Tabata, Tokyo (JP); Ayako Noguchi, Tsukuba-shi (JP)

Correspondence Address: NIXON & VANDERHYE, PC, 1100 N GLEBE ROAD, 8TH FLOOR, ARLINGTON, VA, 22201-4714, US

(73) Assignee: KYOWA HAKKO KOGYO CO., LTD., Tokyo (JP)

(21) Appl. No.: 10/995,266

(22) Filed: Nov. 24, 2004

(30) Foreign Application Priority Data
Nov. 27, 2003 (JP) 2003-397104
Jun. 25, 2004 (JP) 2004-189009

(51) Int. Cl. C12P 21/06; C12N 9/54

(52) U.S. Cl. 435/68.1; 435/221

(57) ABSTRACT

The present invention provides a process for producing a dipeptide which comprises: allowing an enzyme source and one or more kinds of substances selected from the group consisting of amino acid amides, amino acid esters and amino acids to be present in an aqueous medium, said enzyme source being a culture of a microorganism having the ability to produce a dipeptide or a treated matter of the culture, and having been subjected to heat treatment at a temperature of 41 to 65° C. for 30 seconds to one hour so as to produce and accumulate the dipeptide in the aqueous medium; and recovering the dipeptide from the aqueous medium.

### BACKGROUND OF THE INVENTION

The present invention relates to a process for producing a dipeptide using a microorganism producing a dipeptide.

Dipeptides are compounds that are important as foods, pharmaceuticals, cosmetics and the like.

Known methods for producing dipeptides include methods which comprise causing certain kinds of microorganisms or proline iminopeptidase to act on L-amino acid esters and L-amino acids (see WO03/010189 pamphlet and WO03/010307 pamphlet) and a method which comprises causing L-amino acid amide hydrolase to act on L-amino acid asides and L-amino acids (see WO03/010187 pamphlet).

According to the above methods, better production cost and efficiency are achieved by using, as an enzyme source, the cells of a microorganism which produces L-amino acid amide hydrolase or proline iminopeptidase rather than isolated L-amino acid amide hydrolase or proline iminopeptidase. However, use of microbial cells as an enzyme source involves a problem that the formed dipeptide is decomposed by peptidase possessed by the microorganism. Microorganisms have various dipeptidase activities as evidenced, for example, by the fact that more than 20 genes that are presumed to be peptidases of Escherichia coli, a typical microorganism, are registered with the database of DNA Data Bank of Japan (DDBJ).

Therefore, when a dipeptide is produced by using a culture of a microorganism having the ability to produce a dipeptide or a treated matter thereof as an enzyme source, it is considered that the reaction to decompose the dipeptide occurs simultaneously with the reaction to form the dipeptide, leading to remarkably poor yield of the dipeptide. In WO03/010307 pamphlet, solution of this problem is attempted by the addition of a metal enzyme inhibitor. However, productivity of the dipeptide is not sufficient.

An object of the present invention is to provide a process for producing a dipeptide using a microorganism producing a dipeptide.

### SUMMARY OF THE INVENTION

The present invention relates to the following (1) to (16).
(1) A process for producing a dipeptide, which comprises: allowing an enzyme source and one or more kinds of substances selected from the group consisting of amino acid amides, amino acid esters and amino acids to be present in an aqueous medium, said enzyme source being a culture of a microorganism having the ability to produce a dipeptide or a treated matter of the culture, and having been subjected to heat treatment at a temperature of 41 to 65° C. for 30 seconds to one hour so as to produce and accumulate the dipeptide in the aqueous medium; and recovering the dipeptide from the aqueous medium.
(2) The process according to the above (1), wherein the microorganism having the ability to produce a dipeptide is a microorganism belonging to the genus Achromobacter, Acinetobacter, Aeromonas, Agrobacterium, Alcaligenes, Arthrobacter, Beijerinckia, Brevibacterium, Clavibacter, Chryseobacterium, Escherichia, Enterobacter, Erwinia, Flavobacterium, Kluyvera, Microbacterium, Micrococcus, Mycoplana, Pantoea, Propionibacterium, Listonella, Rhizobium, Rhodococcus, Salmonella, Sarcina, Serratia, Staphylococcus, Stenotrophomonas, Streptomyces, Vibrio, Xanthomonas, Bullera, Candida, Cryptococcus, Filobasidium, Geotrichum, Pachysolen, Rhodosporidium, Rhodotorula, Saccharomyces, Sporobolomyces, Tremella, Torulaspora, Torulopsis, Gluconacetobacter, Acetobacter, Gluconobacter, Asaia, Zucharibacter, Actinomadura, Kitasatosporia, Micromonospora, Nocardia, Oerskovia, Saccharothrix, Streptoverticillium, Hafnia, Lactobacillus, Neisseria, Thermoplasma, Corynebacterium, Pseudomonas, Bacillus, Trichosporon or Sterigmatomyces.
[3] The process according to the above (1) or (2), wherein the microorganism having the ability to produce a dipeptide is a microorganism having the ability to produce a protein according to any of [1] to [4] below:
   [1] a protein having the amino acid sequence shown in any of SEQ ID NOS: 1 to 7 and 35;
   [2] a protein consisting of an amino acid sequence wherein one or more amino acid residues are deleted, substituted or added in the amino acid sequence shown in any of SEQ ID NOS: 1 to 7 and 35 and having the activity to synthesize a dipeptide;
   [3] a protein consisting of an amino acid sequence which has at least 65% or more, preferably at least 80% or more, more preferably at least 90% or more, even more preferably at least 95% or more, further preferably at least 98% or more and most preferably at least 99% or more homology to the amino acid sequence shown in any of SEQ ID NOS: 1 to 7 and 35 and having the activity to synthesize a dipeptide; and
   [4] a protein comprising an amino acid sequence which has at least 80% or more, preferably at least 90% or more, more preferably at least 95% or more, even more preferably at least 98% or more and further preferably at least 99% or more homology to the amino acid sequence shown in SEQ ID NO: 15 and having the activity to synthesize a dipeptide.
(4) The process according to the above (1) or (2), wherein the microorganism having the ability to produce a dipeptide is a microorganism carrying DNA according to any of [1] to [4] below:
   [1] DNA encoding the protein according to any of [1] to (4) of the above (3);
   [2] DNA having the nucleotide sequence shown in any of SEQ ID NOS: 8 to 14, 29 and 30;
   [3] DNA which hybridizes with DNA having the nucleotide sequence shown in any of SEQ ID NOS: 8 to 14, 29 and 30 under stringent conditions and which encodes a protein having the activity to synthesize a dipeptide; and
   [4] DNA comprising a nucleotide sequence which has at least 80% or more, preferably at least 90% or more, more preferably at least 95% or more, even more preferably at least 98% or more and further preferably at least 99% or more homology to the nucleotide sequence shown in SEQ ID NO: 16 and encoding a protein having the activity to synthesize a dipeptide.
(5) The process according to the above (1) or (2), wherein the microorganism having the ability to produce a dipeptide is a microorganism producing a protein having proline iminopeptidase [EC 34.11.5] activity or a protein having L-amino acid amide hydrolase activity.
(6) The process according to the above (5), wherein the protein having proline iminopeptidase activity is a protein according to any of [1] to [3] below:
   [1] a protein having the amino acid sequence shown in any of SEQ ID NOS: 17 to 19;
   [2] a protein consisting of an amino acid sequence wherein one or more amino acid residues are deleted, substituted or added in the amino acid sequence shown in any of SEQ ID NOS: 17 to 19 and having proline iminopeptidase activity; and
   [3] a protein consisting of an amino acid sequence which has at least 80% or more, preferably at least 90% or more, more preferably at least 95% or more, even more preferably at least 98% or more and further preferably at least 99% or more homology to the amino acid sequence shown in any of SEQ ID NOS: 17 to 19 and having proline iminopeptidase activity.)
(7) The process according to the above (5), wherein the microorganism producing a protein having proline iminopeptidase activity is a microorganism carrying DNA according to any of [1] to [3] below:
   [1] DNA encoding the protein according to any of [1] to [3] of the above (6);
   [2] DNA having the nucleotide sequence shown in any of SEQ ID NOS: 20 to 22; and
   [3] DNA which hybridizes with DNA having the nucleotide sequence shown in any of SEQ ID NOS: 20 to 22 under stringent conditions and which encodes a protein having proline iminopeptidase activity
(8) The process according to the above (5), wherein the protein having L-amino acid amide hydrolase activity is a protein according to any of [1] to [3] below:
   [1] a protein having the amino acid sequence shown in SEQ ID NO: 23;
   [2] a protein consisting of an amino acid sequence wherein one or more amino acid residues are deleted, substituted or added in the amino acid sequence shown in SEQ ID NO: 23 and having L-amino acid amide hydrolase activity; and
   [3] a protein consisting of an amino acid sequence which has at least 80% or more, preferably at least 90% or more, more preferably at least 95% or more, even more preferably at least 98% or more and further preferably at least 99% or more homology to the amino acid sequence shown in SEQ ID NO: 23 and having L-amino acid amide hydrolase activity.
(9) The process according to the above (5), wherein the microorganism producing a protein having L-amino acid amide hydrolase activity is a microorganism carrying DNA according to any of [1] to [3] below:
   [1] DNA encoding the protein according to any of [1] to [3] of the above (8);
   [2] DNA having the nucleotide sequence shown in SEQ ID NO: 24; and
   [3] DNA which hybridizes with DNA having the nucleotide sequence shown in SEQ ID NO: 24 under stringent conditions and which encodes a protein having L-amino acid amide hydrolase activity.
(10) The process according to the above (1), wherein the microorganism having the ability to produce a dipeptide is a microorganism carrying a recombinant DNA in which the DNA according to any of [1] to [4] of the above (4), [1] to [3] of the above (7) and [1] to [3] of the above (9) is ligated to a vector DNA.
(11) The process according to the above (10), wherein the microorganism carrying a recombinant DNA is a microorganism belonging to the genus Escherichia, Bacillus, Pseudomonas, Corynebacterium or Saccharomyces.
(12) The process according to any of the above (1) to (11), wherein the amino acid is an amino acid selected from the group consisting of L-amino acids, glycine and β-alanine.
(13) The process according to the above (12), wherein the L-amino acid is one or more kinds of amino acids selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine and L-6-diazo-5-oxo-norleucine.
(4) The process according to any of the above (1), (2), (5) to (7), (10) and (11), wherein the amino acid ester is one or more kinds of amino acid esters selected from the group consisting of L-alanine ester, glycine ester, L-valine ester, L-isoleucine ester, L-methionine ester, L-phenylalanine ester, L-serine ester, L-threonine ester, L-glutamine ester, L-tyrosine ester, L-arginine ester, L-aspartic acid-α-ester, L-aspartic acid-β-ester, L-leucine ester, L-asparagine ester, L-lysine ester, L-aspartic acid-α,β-dimethyl ester and L-glutamine-γ-ester, and the amino acid is one or more kinds of amino acids selected from the group consisting of L-glutamic acid, L-glutamine, L-asparagine, glycine, L-alanine, L-leucine, L-methionine, L-proline, L-phenylalanine, L-tryptophan, L-serine, L-threonine, L-tyrosine, L-lysine, L-arginine and L-histidine.
(15) The process according to any of the above (1), (2), (5) and (8) to (11), wherein the amino acid amide is one or more kinds of amino acid amides selected from the group consisting of L-alanine amide, glycine amide and L-aspartic acid-α-amide, and the amino acid is one or more kinds of amino acids selected from the group consisting of L-glutamic acid, L-asparagine, L-glutamine, glycine, L-alanine, L-valine, L-leucine, L-isoleucine, L-methionine, L-proline, L-phenylalanine, L-tryptophan, L-serine, L-threonine, L-tyrosine, L-lysine, L-arginine and L-histidine.
(16) The process according to any of the above (1) to (15), wherein the treated matter of the culture is concentrated culture, dried culture, cells obtained by centrifuging the culture, or a product obtained by subjecting the cells to drying, freeze-drying, treatment with a surfactant, treatment with a solvent, enzymatic treatment or immobilization.

In accordance with the present invention, there is provided a process for producing a dipeptide using a culture of a microorganism or a treated matter thereof.

### BRIEF DESCRIPTION OF THE DRAWINGS

FIG. 1 shows the steps for constructing His-tagged ywfE expression vector pQE60ywfE.

### EXPLANATION OF SYMBOL

PT5: T5 promoter

### DETAILED DESCRIPTION OF THE INVENTION

1. Microorganisms Used in the Process for Producing a Dipeptide of the Present Invention

There is not any specific restriction as to the microorganism used in the process for producing a dipeptide of the present invention, so long as it has the ability to produce a dipeptide from one or more kinds of substances selected from the group consisting of L-amino acid amides, L-amino acid esters and amino acids.

Suitable microorganisms include those belonging to the genera Achromobacter, Acinetobacter, Aeromonas, Agrobacterium, Alcaligenes, Arthrobacter, Beijerinckia, Brevibacterium, Clavibacter, Chryseobacterium, Escherichia, Enterobacter, Erwinia, Flavobacterium, Kluyvera, Microbacterium, Micrococcus, Mycoplana, Pantoea, Propionibacterium, Listonella, Rhizobium, Rhodococcus, Salmonella, Sarcina, Serratia, Staphylococcus, Stenotrophomonas, Streptomyces, Vibrio, Xanthomonas, Bullera, Candida, Cryptococcus, Filobasidium, Geotrichum, Pachysolen, Rhodosporidium, Rhodotorula, Saccharomyces, Sporobolomyces, Tremella, Torulaspora, Torulopsis, Gluconacetobacter, Acetobacter, Gluconobacter, Asaia, Zucharibacter, Actinomadura, Kitasatosporia, Micromonospora, Nocardia, Oerskovia, Saccharothrix, Streptoverticillium, Hafnia, Lactobacillus, Neisseria, Thermoplasma, Corynebacterium, Pseudomonas, Bacillus, Trichosporon and Sterigmatomyces.

Examples of one or more kinds of substances selected from the group consisting of amino acid amides, amino acid esters and amino acids are one or more kinds of amino acids; one or more kinds of amino acid esters and one or more kinds of amino acids; and one or more kinds of amino acid amides and one or more kinds of amino acids.

(1) Microorganisms Having the Ability to Produce a Dipeptide from One or More Kinds of Amino Acids

The microorganisms having the ability to produce a dipeptide from one or more kinds of amino acids may be any microorganisms having such ability and include microorganisms producing a protein selected from the group consisting of non-ribosomal peptide synthetase (hereinafter referred to as NRPS), D-alanyl-D-alanine (D-Ala-D-Ala) ligase and bacilysin synthetase. Examples of the microorganisms producing NRPS include procaryotes such as microorganisms of the genus Bacillus, microorganisms producing BacA, BacB and BacC (GenBank AF007865), microorganisms producing TycA, TycB and TycC (GenBank AF004835), microorganisms producing PcbAB (GenBank M57425), and microorganisms producing a protein having an amino acid sequence which has at least 80% or more, preferably at least 90% or more, more preferably at least 95% or more, even more preferably at least 98% or more and further preferably at least 99% or more homology to the amino acid sequence of any protein selected from BacA, BacB, BacC, TycA, TycB, TycC and PcbAB and having NRPS activity.

Examples of the microorganisms producing D-Ala-D-Ala ligase include procaryotes forming peptidoglycans, microorganisms producing Dd1A (GenBank accession No. M58467), microorganisms producing Dd1B (GenBank accession No. AE000118), microorganisms producing Dd1C (GenBank accession No. D88151), and microorganisms producing a protein consisting of an amino acid sequence wherein one or more amino acid residues are deleted, substituted or added in the amino acid sequence of any protein selected from Dd1A, Dd1B and Dd1C and having D-Ala-D-Ala ligase activity.

Examples of the microorganisms producing bacilysin synthetase include microorganisms belonging to the genus Bacillus, preferably, Bacillus subtilis, Bacillus amyloliquefaciens, Bacillus coagulans, Bacillus licheniformis, Bacillus megaterium and Bacillus pumilus, more preferably, Bacillus subtilis 168 (ATCC 23857), Bacillus subtilis ATCC 15245, Bacillus subtilis ATCC 6633, Bacillus subtilis IAM 1213, Bacillus subtilis IAM 1107, Bacillus subtilis IAM 1214, Bacillus subtilis ATCC 9466, Bacillus subtilis IAM 1033, Bacillus subtilis ATCC 21555 and Bacillus amyloliquefaciens IFO 3022, and microorganisms producing a protein selected from the following [1] to [4]:
[1] a protein having the amino acid sequence shown in any of SEQ ID NOS: 1 to 7 and 35;
[2] a protein consisting of an amino acid sequence wherein one or more amino acid residues are deleted, substituted or added in the amino acid sequence shown in any of SEQ ID NOS: 1 to 7 and 35 and having the activity to synthesize a dipeptide;
[3] a protein consisting of an amino acid sequence which has at least 65% or more, preferably at least 80% or more, more preferably at least 90% or more, even more preferably at least 95% or more, further preferably at least 98% or more and most preferably at least 99% or more homology to the amino acid sequence shown in any of SEQ ID NOS: 1 to 7 and 35 and having the activity to synthesize a dipeptide; and
[4] a protein comprising an amino acid sequence which has 80% or more, preferably at least 90% or more, more preferably at least 95% or more, even more preferably at least 98% or more and further preferably at least 99% or more homology to the amino acid sequence shown in SEQ ID NO: 15 and having the activity to synthesize a dipeptide.

The amino acid sequence shown in SEQ ID NO: 15 is a region conserved among the proteins having the amino acid sequences shown in SEQ ID NOS: 1 to 7 and is also a region corresponding to the consensus sequence of proteins having Ala-Ala ligase activity derived from various microorganisms.

Therefore, microorganisms producing a protein comprising an amino acid sequence which has at least 80% or more, preferably 90% or more, more preferably 95% or more, even more preferably at least 98% or more and further preferably at least 99% or more homology to the amino acid sequence shown in SEQ ID NO: 15 and having the activity to synthesize a dipeptide are also included in the dipeptide-producing microorganisms.

In order that the protein comprising an amino acid sequence which has at least 80% or more, preferably 90% or more, more preferably at least 95% or more, even more preferably at least 98% or more and further preferably at least 99% or more homology to the amino acid sequence shown in SEQ ID NO: 15 may have the activity to synthesize a dipeptide, it is desirable that the homology of its amino acid sequence to the amino acid sequence shown in any of SEQ ID NOS: 1 to 7 is at least 65% or more, preferably at least 80% or more, more preferably at least 90% or more, even more preferably at least 95% or more, further preferably at least 98% or more and most preferably at least 99% or more.

(2) Microorganisms Having the Ability to Produce a Dipeptide from One or More Kinds of Amino Acid Esters and One or More Kinds of Amino Acids

The microorganisms having the ability to produce a dipeptide from one or more kinds of amino acid esters and one or more kinds of amino acids may be any microorganisms having such ability and include those belonging to the genera Achromobacter, Acinetobacter, Aeromonas, Agrobacterium, Alcaligenes, Arthrobacter, Beijerinckia, Brevibacterium, Clavibacter, Chryseobacterium, Escherichia, Enterobacter, Erwinia, Flavobacterium, Kluyvera, Microbacterium, Micrococcus, Mycoplana, Pantoea, Propionibacterium, Listonella, Rhizobium, Rhodococcus, Salmonella, Sarcina, Serratia, Staphylococcus, Stenotrophomonas, Streptomyces, Vibrio, Xanthomonas, Bullera, Candida, Cryptococcus, Filobasidium, Geotrichum, Pachysolen, Rhodosporidium, Rhodotorula, Saccharomyces, Sporobolomyces, Tremella, Torulaspora, Torulopsis, Gluconacetobacter, Acetobacter, Gluconobacter, Asaia, Zucharibacter, Actinomadura, Kitasatosporia, Micromonospora, Nocardia, Oerskovia, Saccharothrix, Streptoverticillium, Hafnia, Lactobacillus, Neisseria, Thermoplasma, Corynebacterium, Pseudomonas and Bacillus.

Specific examples of the above microorganisms include Achromobacter delmarvae (FERM BP-6988), Acinetobacter johnsonii (ATCC 9036), Aeromonas salmonicida (ATCC 14174), Agrobacterium tumefaciens (IFO 3058), Alcaligenes faecalis (ATCC 8750), Arthrobacter citreus (ATCC 11624), Beijerinckia indica (ATCC 9037), Brevibacterium roseum (ATCC 13825), Clavibacter michiganense (ATCC 7429), Chryseobacterium meningosepticum (ATCC 13253), Escherichia coli (ATCC 13071), Enterobacter aerogenes (ATCC 13048), Erwinia amylovora (IFO 12687), Flavobacterium resinovorum (ATCC 12524), Kluyvera citrophila (FERM BP-6564), Microbacterium imperiale (ATCC 8365), Micrococcus luteus (ATCC 11880), Mycoplana bullata (ATCC 4278), Pantoea ananatis (ATCC 23822), Propionibacterium shermanii (FERM BP-8100), Listonella anquillarum (ATCC 19264), Rhizobium radiobacter (ATCC 4720), Rhodococcus rhodochrous (ATCC 21198), Salmonella typhimurium (FERM BP-6566), Sarcina lutea (FERM BP-6562), Serratia grimesii (ATCC 14460), Staphylococcus aureus (ATCC 12600), Stenotrophomonas maltophilia (ATCC 13270), Streptomyces lavendulae (ATCC 11924), Vibrio tyrogenes (FERM BP-5848), Xanthomonas maltophilia (FERM BP-5568), Bullera alba (FERM BP-8099), Candida krusei (IFO 0011), Cryptococcus terreus (IFO 0727), Filobasidium capsuligenum (IFO 1119), Geotrichum amycelicum (ATCC 56046), Pachysolen tannophilus (IFO 1007), Rhodosporidium diobovatum (IFO 1829), Rhodotorula minuta (IFO 0879), Saccharomyces unisporus (IFO 0724), Sporobolomyces salmonicolor (IFO 1038), Tremella foliacea (IFO 9297), Torulaspora delbrueckii (IFO 1083), Torulopsis ingeniosa (FERM BP-8098), Gluconacetobacter liquefaciens (IFO 12388), Acetobacter orleanensis (IFO 3223), Acetobacter pasteurianus (ATCC 9325), Gluconobacter oxydans (ATCC 621), Gluconobacter oxydans (IFO 3171), Gluconacetobacter hansenii (JCM 7643), Asaia ethanolifaciens (FERM BP-6751), Zucharibacter floricola (FREM BP-6752), Actinomadura madurae (ATCC 19425), Kitasatosporia griseola (IFO 14371), Micromonospora chersina (ATCC 53710), Nocardia globerula (ATCC 21602), Oerskovia turbata (FERM BP-8122), Saccharothrix australiensis (IFO 14444), Streptoverticillium mobaraensis (IFO 13819), Streptomyces plicatus [Biochem. Biophys. Res. Commun., 184, 1250 (1992)], Corynebacterium variabilis [J. Appl. Microbiol., 90, 449 (2001)], Arthrobacter nicotianae [FEMS Microbiol. Lett., 78, 191 (1999)], Escherichia coli (Japanese Published Unexamined Patent Application No. 113887/90), Flavobacterium meningosepticum [Arch. Biochem. Biophys., 336, 35 (1996)], Hafnia alvei [J. Biochem., 119, 468 (1996)], Lactobacillus delbrueckii [Microbiology, 140, 527 (1994)], Bacillus coagulans [J. Bacteriol., 174, 7919 (1994)], Aeromonas sobria [J. Biochem., 116, 818 (1994)], Xanthomonas campestris (Japanese Published Unexamined Patent Application No. 121860/97), Neisseria gonorrhoeae [Mol. Microbiol., 9, 1203 (1993)], Propionibacterium freudenreichii [Appl. Environ. Microbiol., 64, 4736 (1998)], Serratia marcescens [J. Biochem., 122, 601 (1997)], Thermoplasma acidophilum [FEBS Lett., 398, 101 (1996)], Pseudomonas aeruginosa [Nature, 406, 959 (2000)], Bacillus subtilis (ATCC 6633), Bacillus coagulans EK01 [J. Bacteriol., 174, 7919 (1992)], Corynebacterium glutamicum (ATCC 13286), Pseudomonas putida (FERM BP-8101), Pseudomonas putida (ATCC 12633) and Pseudomonas putida (FERM BP-8123).

The microorganisms having the ability to produce a dipeptide from one or more kinds of L-amino acid esters and one or more kinds of amino acids also include microorganisms having the ability to produce proline iminopeptidase Examples of such microorganisms are those belonging to the genera Streptomyces, Arthrobacter, Escherichia, Flavobacterium, Hafnia, Lactobacillus, Aeromonas, Xanthomonas, Neisseria, Propionibacterium, Serratia, Thermoplasma, Corynebacterium, Pseudomonas and Bacillus.

Specific examples of the microorganisms are Streptomyces plicatus [Biochem. Biophys. Res. Commun., 184, 1250 (1992)], Arthrobacter nicotianae [FEMS Microbiol. Lett., 78, 191 (1999)), Escherichia coli (Japanese Published Unexamined Patent Application No. 113887/90), Flavobacterium meningosepticum (Arch. Biochem. Biophys., 336, 35 (1996)], Hafnia alvei [J. Biochem., 119, 468 (1996)], Lactobacillus delbrueckii [Microbiology, 140, 527 (1994)], Bacillus coagulans [J. Bacteriol., 174, 7919 (1994)], Aeromonas sobria [J. Biochem., 116, 818 (1994)], Xanthomonas campestris (Japanese Published Unexamined Patent Application No. 121860/97), Neisseria gonorrhoeae [Mol. Microbiol., 9, 1203 (1993)], Propionibacterium freudenreichii [Appl. Environ. Microbiol., 64, 4736 (1998)], Serratia marcescens [J. Biochem., 122, 601 (1997)], Thermoplasma acidophilum [FEBS Lett., 398, 101 (1996)], Pseudomonas aeruginosa [Nature, 406, 959 (2000)], Bacillus subtilis (ATCC 6633), Bacillus coagulans EK01 [J-Bacteriol., 174, 7919 (1992)], Corynebacterium variabilis [J. Appl. Microbiol., 90, 449 (2001)], Corynebacterium glutamicum (ATCC 13286), Pseudomonas putida (FERM BP-8101), Pseudomonas putida (ATCC 12633), Pseudomonas putida (FERM BP-8123) and microorganisms having the ability to produce a protein having the amino acid sequence shown in any of SEQ ID NOS: 17 to 19.

Further, the microorganisms having the ability to produce proline iminopeptidase include microorganisms having the ability to produce a protein of the following [1] or [2]:
[1] proline iminopeptidase derived from Streptomyces plicatus [Biochem. Biophys. Res. Commun., 184, 1250 (1992)], Arthrobacter nicotianae [FEMS Microbiol. Lett., 78, 191 (1999)], Escherichia coli (Japanese Published Unexamined Patent Application No. 113887/90), Flavobacterium meningosepticum [Arch. Biochem. Biophys., 336, 35 (1996)], Hafnia alvei [J. Biochem., 119, 468 (1996)], Lactobacillus delbrueckii [Microbiology, 140, 527 (1994)], Bacillus coagulans [J. Bacteriol., 174, 7919 (1994)], Aeromonas sobria [J. Biochem., 116, 818 (1994)], Xanthomonas campestris (Japanese Published Unexamined Patent Application No. 121860/97), Neisseria gonorrhoeae [Mol. Microbiol., 9, 1203 (1993)], Propionibacterium freudenreichii [Appl. Environ. Microbiol., 64, 4736 (1998)], Serratia marcescens (J. Biochem., 122, 601 (1997)], Thermoplasma acidophilum [FEBS Lett., 398, 101 (1996)] and Pseudomonas aeruginosa (Nature, 406, 959 (2000)], and a protein consisting of an amino acid sequence wherein one or more amino acid residues are deleted, substituted or added in the amino acid sequence shown in any of SEQ ID NOS: 17 to 19 and having proline iminopeptidase activity; and
[2] a protein consisting of an amino acid sequence which has at least 80% or more, preferably at least 90% or more, more preferably at least 95% or more, even more preferably at least 98% or more and further preferably at least 99% or more homology to the amino acid sequence of any of the proline iminopeptidase and the protein of the above [1] and having proline iminopeptidase activity.
   (3) Microorganisms Having the Ability to Produce a Dipeptide from One or More Kinds of L-Amino Acid Amides and One or More Kinds of Amino Acids

The microorganisms having the ability to produce a dipeptide from one or more kinds of L-amino acid amides and one or more kinds of amino acids may be any microorganisms having such ability and include those belonging to the genera Bacillus, Corynebacterium, Erwinia, Rhodococcus, Chryseobacterium, Micrococcus, Pseudomonas, Cryptococcus, Trichosporon, Rhodosporidium, Sporobolomyces, Tremella, Torulaspora, Sterigmatomyces and Rhodotorula.

Specific examples of the microorganisms are Bacillus megaterium (FERM BP-8090), Corynebacterium glutamicum (ATCC 13286), Erwinia carotovora (FERM BP-8089), Rhodococcus rhodochrous (ATCC 19149), Chryseobacterium meningosepticum (ATCC 13253), Micrococcus luteus (ATCC 9341), Pseudomonas saccharophila (ATCC 15946), Cryptococcus albidus (IFO 0378), Trichosporon gracile (ATCC 24660), Rhodosporidium diobovatum (ATCC 22264), Sporobolomyces salmonicolor (IFO 1038), Tremella foliacea (IFO 9297), Torulaspora delbrueckii (IFO 1083), Sterigmatomyces elviae (IFO 1843) and Rhodotorula ingeniosa (ATCC 22993).

The microorganisms having the ability to produce a dipeptide from one or more kinds of L-amino acid amides and one or more kinds of amino acids also include microorganisms having the ability to produce L-amino acid amide hydrolase, and examples of the microorganisms are Corynebacterium glutamicum (ATCC 13286) and those having the ability to produce a protein having the amino acid sequence shown in SEQ ID NO: 23.

Further, the microorganisms having the ability to produce L-amino acid amide hydrolase include microorganisms having the ability to produce a protein of the following [1] or [2]:
[1] a protein consisting of an amino acid sequence wherein one or more amino acid residues are deleted, substituted or added in the amino acid sequence shown in SEQ ID NO: 23 and having L-amino acid amide hydrolase activity; and
[2] a protein consisting of an amino acid sequence which has at least 80% or more, preferably at least 90% or more, more preferably at least 95% or more, even more preferably at least 98% or more and further preferably at least 99% or more homology to the amino acid sequence shown in SEQ ID NO: 23 and having L-amino acid amide hydrolase activity.

The above protein consisting of an amino acid sequence wherein one or more amino acid residues are deleted, substituted or added and having the activity to synthesize a dipeptide can be obtained, for example, by introducing a site-directed mutation into DNA encoding a protein selected from a protein consisting of the amino acid sequence shown in any of SEQ ID NOS: 1 to 7 and 35, a protein consisting of the amino acid sequence shown in any of SEQ ID NOS: 17 to 19 and having proline iminopeptidase activity, and a protein consisting of the amino acid sequence shown in SEQ ID NO: 23 and having L-amino acid amide hydrolase activity by site-directed mutagenesis described in Molecular Cloning, A Laboratory Manual, Second Edition, Cold Spring Harbor Laboratory Press (1989) (hereinafter referred to as Molecular Cloning, Second Edition); Current Protocols in Molecular Biology, John Wiley & Sons (1987-1997) (hereinafter referred to as Current Protocols in Molecular Biology); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc.

The number of amino acid residues which are deleted, substituted or added is not specifically limited so long as it is within the range where deletion, substitution or addition is possible by known methods such as the above site-directed mutagenesis. The suitable number is 1 to dozens, preferably 1 to 20, more preferably 1 to 10, further preferably 1 to 5.

The expression “one or more amino acid residues are deleted, substituted or added in the amino acid sequence of any of a protein consisting of the amino acid sequence shown in any of SEQ ID NOS: 1 to 7 and 35, a protein consisting of the amino acid sequence shown in any of SEQ ID NOS: 17 to 19 and having proline iminopeptidase activity, and a protein consisting of the amino acid sequence shown in SEQ ID NO. 23 and having L-amino acid amide hydrolase activity” means that the amino acid sequence may contain deletion, substitution or addition of a single or plural amino acid residues at an arbitrary position therein.

Deletion, substitution and addition may be simultaneously contained in one sequence, and amino acids to be substituted or added may be either natural or not Examples of the natural amino acids are L-alanine, L-asparagine, L-aspartic acid, L-glutamine, L-glutamic acid, glycine, L-arginine, L-histidine, L-isoleucine, L-leucine, L-lysine, L-methionine, L-phenylalanine, L-proline, L-serine, L-threonine, L-tryptophan, L-tyrosine, L-valine and L-cysteine.

The following are examples of the amino acids capable of mutual substitution. The amino acids in the same group can be mutually substituted.
Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2-aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

There is not any specific restriction as to the position where the above deletion, substitution or addition of one or more amino acid residues is introduced, so long as a protein having an amino acid sequence carrying the introduced mutation has the dipeptide-synthesizing activity. Suitable examples include amino acid residues which are not conserved in all of the amino acid sequences shown in SEQ ID NOS: 1 to 7 and 35 when the sequences are compared using known alignment software. An example of known alignment software is alignment analysis software contained in gene analysis software Genetyx (Software Development Co., Ltd.). As analysis parameters for the analysis software, default values can be used.

The above proteins consisting of an amino acid sequence wherein one or more amino acid residues are deleted, substituted or added and having the activity to synthesize a dipeptide include proteins consisting of an amino acid sequence which has at least 65% or more, preferably at least 80% or more, more preferably at least 90% or more, even more preferably at least 95% or more, further preferably at least 98% or more and most further preferably at least 99% or more homology to the amino acid sequence shown in any of SEQ ID NOS: 1 to 7 and 35, the amino acid sequence shown in any of SEQ ID NOS: 17 to 19 or the amino acid sequence shown in SEQ ID NO: 23.

The above homology among amino acid sequences and nucleotide sequences can be determined by using algorithm BLAST by Karlin and Altschul [Proc. Natl. Acad. Sci. USA, 90, 5873 (1993)] and FASTA [Methods Enzymol., 183, 63 (1990)]. On the basis of the algorithm BLAST, programs such as BLASTN and BLASTX have been developed [J. Mol. Biol., 215, 403 (1990)]. When a nucleotide sequence is analyzed by BLASTN on the basis of BLAST, the parameters, for instance, are as follows: score-100 and wordlength=12. When an amino acid sequence is analyzed by BLASTX on the basis of BLAST, the parameters, for instance, are as follows: score=50 and wordlength=3. When BLAST and Gapped BLAST programs are used, default parameters of each program are used. The specific techniques for these analyses are known (http://www.ncbi.nlm.nih.gov.).

(4) Microorganisms Transformed with DNA Encoding a Protein Having the Activity to Synthesize a Dipeptide

The microorganisms transformed with DNA encoding a protein having the activity to synthesize a dipeptide include those carrying a recombinant DNA obtained by ligating the DNA to a vector DNA.

Examples of the microorganisms are those carrying a recombinant DNA obtained by ligating, to a vector DNA, DNA encoding a protein having the activity to synthesize a dipeptide from one or more kinds of amino acids, DNA encoding a protein having proline iminopeptidase activity or DNA encoding a protein having L-amino acid amide hydrolase activity.

Examples of the microorganisms include those belonging to the genera Escherichia, Bacillus, Corynebacterium, Pseudomonas and Saccharomyces.

The DNAs encoding a protein having the activity to synthesize a dipeptide from one or more kinds of amino acids include DNAs encoding NRPS, D-Ala-D-Ala ligase or bacilysin synthetase.

Examples of the DNAs encoding NRPS include DNAs encoding a protein selected from the group consisting of BacA, BacB, BacC, TycA, TycB, TycC and PcbAB.

Examples of the DNAs encoding D-Ala-D-Ala ligase include DNAs encoding a protein selected from the group consisting of Dd1A, Dd1B and Dd1C.

Examples of the DNAs encoding bacilysin synthetase include DNAs encoding a protein of any of the following [1] to [4]:
[1] a protein having the amino acid sequence shown in any of SEQ ID NOS: 1 to 7 and 35;
[2] a protein consisting of an amino acid sequence wherein one or more amino acid residues are deleted, substituted or added in the amino acid sequence shown in any of SEQ ID NOS: 1 to 7 and 35 and having the activity to synthesize a dipeptide;
[3] a protein consisting of an amino acid sequence which has at least 80% or more, preferably at least 90% or more, more preferably at least 95% or more, even more preferably at least 98% or more and further preferably at least 99% or more homology to the amino acid sequence shown in any of SEQ ID NOS: 1 to 7 and 35 and having the activity to synthesize a dipeptide; and
[4] a protein comprising an amino acid sequence which has at least 80% or more, preferably at least 90% or more, more preferably at least 95% or more, even more preferably at least 98% or more and further preferably at least 99% or more homology to the amino acid sequence shown in SEQ ID NO: 15 and having the activity to synthesize a dipeptide;
   and DNAs of the following [51 to [7]:
[5] DNA having the nucleotide sequence shown in any of SEQ ID NOS: 8 to 14, 29 and 30;
[6] DNA which hybridizes with DNA having the nucleotide sequence shown in any of SEQ ID NOS: 8 to 14, 29 and 30 under stringent conditions and which encodes a protein having the activity to synthesize a dipeptide; and
[7] DNA comprising a nucleotide sequence which has at least 80% or more, preferably at least 90% or more, more preferably at least 95% or more, even more preferably at least 98% or more and further preferably at least 99% or more homology to the nucleotide sequence shown in SEQ ID NO: 16 and encoding a protein having the activity to synthesize a dipeptide.

Examples of the DNAs encoding a protein having proline iminopeptidase activity include DNAs encoding proline iminopeptidase or a protein of any of the following [1] to [3]:
[1] proline iminopeptidase described in any of Biochem. Biophys. Res. Commun., 184, 1250 (1992); FEMS Microbiol. Lett., 78, 191 (1999); Japanese Published Unexamined Patent Application No. 113887/90; Arch. Biochem. Biophys., 336, 35 (1996); J. Biochem., 119, 468 (1996); Microbiology, 140, 527 (1994); J. Bacteriol., 174, 7919 (1994); J. Biochem., 116, 818 (1994); Japanese Published Unexamined Patent Application No. 121860/97; Mol. Microbiol., 9, 1203 (1993); Appl. Environ. Microbiol., 64, 4736 (1998); J. Biochem., 122, 601 (1997); FEBS Lett., 398, 101 (1996); and Nature, 406, 959 (2000), and a protein having the amino acid sequence shown in any of SEQ ID NOS: 17 to 19;
[2] a protein consisting of an amino acid sequence wherein one or more amino acid residues are deleted, substituted or added in the amino acid sequence of any of the proline iminopeptidase or the protein of the above [1] and having proline iminopeptidase activity; and
[3] a protein consisting of an amino acid sequence which has at least 80% or more, preferably at least 90% or more, more preferably at least 95% or more, even more preferably at least 98% or more and further preferably at least 99% or more homology to the amino acid sequence of any of the proline iminopeptidase or the protein of the above [1] and having proline iminopeptidase activity;
and DNAs of the following [4] and [5]:
[4] DNA encoding proline iminopeptidase described in any of Biochem. Biophys. Res. Commun., 184, 1250 (1992); FEMS Microbiol. Lett., 78, 191 (1999); Japanese Published Unexamined Patent Application No. 113887/90; Arch. Biochem. Biophys., 336, 35 (1996); J. Biochem., 119, 468 (1996); Microbiology, 140, 527 (1994); J. Bacteriol., 174, 7919 (1994); J. Biochem., 116, 818 (1994); Japanese Published Unexamined Patent Application No. 121860/97; Mol. Microbiol., 9, 1203 (1993); Appl. Environ. Microbiol., 64, 4736 (1998); J. Biochem., 122, 601 (1997); FEBS Lett., 398, 101 (1996); and Nature, 406, 959 (2000), and DNA having the nucleotide sequence shown in any of SEQ ID NOS: 20 to 22; and
[5] DNA which hybridizes with any DNA of the above 14] under stringent conditions and which encodes a protein having proline iminopeptidase activity.

Examples of the DNAs encoding a protein having L-amino acid amide hydrolase activity include DNAs encoding a protein of any of the following [1] to [3]:
[1] a protein having the amino acid sequence shown in SEQ ID NO: 23;
[2] a protein consisting of an amino acid sequence wherein one or more amino acid residues are deleted, substituted or added in the amino acid sequence shown in SEQ ID NO: 23 and having L-amino acid amide hydrolase activity; and
[3] a protein consisting of an amino acid sequence which has at least 80% or more, preferably at least 90% or more, more preferably at least 95% or more, even more preferably at least 98% or more and further preferably at least 99% or more homology to the amino acid sequence shown in SEQ ID NO: 23 and having L-amino acid amide hydrolase activity;
and DNAs of the following [4] and [5]:
[4] DNA having the nucleotide sequence shown in SEQ ID NO: 24 and encoding L-amino acid amide hydrolase; and
[5] DNA which hybridizes with DNA consisting of the nucleotide sequence shown in SEQ ID NO: 24 and encoding L-amino acid amide hydrolase under stringent conditions and which encodes a protein having L-amino acid amide hydrolase activity.

The term “hibridizing” as used herein refers to a process in which polynucleotides hybridize to the recited nucleic acid sequence or parts thereof. Therefore, said nucleic acid sequence may be useful as probes in Northern or Southern Blot analysis of RNA or DNA preparations, respectively, or can be used as oligonucleotide primers in PCR analysis dependent on their respective size. Preferably, said hybridizing polynucleotides comprise at least 10, more preferably at least 15 nucleotides while a hybridizing polynucleotide of the present to be used as a probe preferably comprises at least 100, more preferably at least 200, or most preferably at least 500 nucleotides.

It is well known in the art how to perform hybridization experiments with nucleic acid molecules, i.e. the person skilled in the art knows what hybridization conditions s/he has to use in accordance with the present invention. Such hybridization conditions are referred to in standard text books such as Sambrook et al.; Molecular Cloning: A Laboratory Manual; Cold Spring Harbor Laboratory Press, 2nd edition 1989 and 3rd edition 2001; Gerhardt et al.; Methods for General and Molecular Bacteriology; ASM Press, 1994; Lefkovits; Immunology Methods Manual: The Comprehensive Sourcebook of Techniques; Academic Press, 1997; Golemis; Protein-Protein Interactions: A Molecular Cloning Manual; Cold Spring Harbor Laboratory Press, 2002 and other standerd laboratory manuals known by the person skilled in the art or as recited above. Preferred in accordance with the present inventions are stringent hybridization conditions.

“Stringent hybridization conditions” refer, e.g. to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed e.g. by washing the filters in 0.2×SSC at about 65° C. Also contemplated are nucleic acid molecules that hybridize at low stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6×SSPE (20×SSPE=3 mol/l NaCl; 0.2 mol/l NaH2PO4; 0.02 mol/l EDTA, pH 7.4), 0.5% SDS, 30% formamide, 100 μ/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). It is of note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of hybridization conditions described above, due to problems with compatibility.

The hybridizable DNA under the above described stringent hybridization conditions includes DNA having at least 80% or more, preferably at least 90% or more, more preferably at least 95% or more, even more preferably at least 98% or more, further preferably at least 99% or more homology to the nucleotide sequence of any of the DNAs described above as calculated by use of programs such as BLAST and FASTA described above based on the above parameters.

The homology among nucleotide sequences can be determined by using a program such as BLAST or FASTA described above.

It is possible to confirm that the DNA hybridizing with the above DNA under stringent conditions is DNA encoding a protein having the activity to synthesize a dipeptide in the following manner. That is, a recombinant DNA expressing the DNA is prepared and the recombinant DNA is introduced into a host cell to obtain a microorganism to be used as an enzyme source. Then, 1) the enzyme source and one or more kinds of amino acids are allowed to be present in an aqueous medium, followed by HPLC analysis or the like to know whether a dipeptide is formed and accumulated in the aqueous medium, 2) the enzyme source, one or more kinds of amino acid esters and one or more kinds of amino acids are allowed to be present in an aqueous medium, followed by HPLC analysis or the like to know whether a dipeptide is formed and accumulated in the aqueous medium, or 3) the enzyme source, one or more kinds of amino acid amides and one or more kinds of amino acids are allowed to be present in an aqueous medium, followed by HPLC analysis or the like to know whether a dipeptide is formed and accumulated in the aqueous medium.

2. Process for Preparing the Microorganisms Used in the Process for Producing a Dipeptide of the Present Invention

The microorganisms used in the process for producing a dipeptide of the present invention include the above-mentioned strains available from various public organizations, strains which are obtained by subjecting the above strains to known mutagenesis and which have the ability to produce a dipeptide, and also recombinant strains in which the ability to produce a dipeptide is intensified by genetic engineering techniques. The recombinant strains include microorganisms in which bacilysin synthetase activity, proline iminopeptidase activity or L-amino acid amide hydrolase activity is enhanced, and examples of the recombinant microorganisms include Escherichia coli NM522/pQE60ywfE, Escherichia coli JM109/pUCAAH (WO03/010187), Escherichia coli JM109/pQEAAH (WO03/010187), Escherichia coli JM 109/pUCPPPEPI (WO03/010307) and Escherichia coli JM 109/pUCPGPEPI (WO03/010307).

(1) Process for Preparing the Recombinant Strains

(a) Acquisition of DNA Encoding a Protein Having the Activity to Synthesize a Dipeptide

The above DNA encoding a protein having the activity to synthesize a dipeptide can be obtained utilizing the nucleotide sequence information on the DNA, for example, by the method described below.

The following illustrates examples of the methods to obtain DNA encoding bacilysin synthetase. That is, the DNA encoding bacilysin synthetase can be obtained, for example, by Southern hybridization of a chromosomal DNA library from a microorganism belonging to the genus Bacillus using a probe designed based on the nucleotide sequence shown in any of SEQ ID NOS: 8 to 14, 29 and 30, or by PCR [PCR Protocols, Academic Press (1990)] using primer DNAs designed based on the nucleotide sequence shown in any of SEQ ID NOS: 8 to 14, 29 and 30 and, as a template, the chromosomal DNA of a microorganism belonging to the genus Bacillus.

The DNA encoding bacilysin synthetase can also be obtained by conducting a search through various gene databases for a sequence having at least 65% or more, preferably at least 80% or more, more preferably at least 90% or more, even more preferably at least 95% or more, further preferably at least 98% or more, most further preferably at least 99% or more homology to the nucleotide sequence of DNA encoding the amino acid sequence shown in any of SEQ ID NOS: 1 to 7, 15 and 35, and obtaining the desired DNA, based on the nucleotide sequence obtained by the search, from a chromosomal DNA or cDNA library of an organism having the nucleotide sequence according to the above-described method.

The obtained DNA, as such or after cleavage with appropriate restriction enzymes, is inserted into a vector by a conventional method to obtain a recombinant DNA. A plasmid DNA is extracted from a transformant obtained by introducing the recombinant DNA into Escherichia coli. Then, the nucleotide sequence of the DNA can be determined by a conventional sequencing method such as the dideoxy method [Proc. Natl. Acad. Sci., USA, 74, 5463 (1977)] or by using a nucleotide sequencer such as 373A DNA Sequencer (Perkin-Elmer Corp.).

In cases where the obtained DNA is found to be a partial DNA by the analysis of nucleotide sequence, the full length DNA can be obtained by Southern hybridization of a chromosomal DNA library using the partial DNA as a probe.

It is also possible to prepare the desired DNA by chemical synthesis using a DNA synthesizer (e.g., Model 8905, PerSeptive Biosystems) based on the determined nucleotide sequence of the DNA.

Examples of the DNAs that can be obtained by the above-described method are DNAs having the nucleotide sequences shown in SEQ ID NOS: 1 to 7, 29 and 30.

Examples of the vectors for inserting the DNA include pBluescript II KS(+) (Stratagene), pDIRECT [Nucleic Acids Res., 18, 6069 (1990)], pCR-Script Amp SK(+) (Stratagene), pT7 Blue (Novagen, Inc.), pCR II (Invitrogen Corp.) and pCR-TRAP (Genhunter Corp.).

Examples of Escherichia coli include Escherichia coli XL1-Blue, Escherichia coli XL2-Blue, Escherichia coli DB1, Escherichia coli MC1000, Escherichia coli KY3276, Escherichia coli W1485, Escherichia coli JM101, Escherichia coli JM109, Escherichia coli HB101, Escherichia coli No. 49, Escherichia coli W3110, Escherichia coli NY49, Escherichia coli MP347, Escherichia coli NM522 and Escherichia coli ME8415.

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

An example of the microorganism carrying the DNA encoding a protein having the dipeptide-synthesizing activity obtained by the above method is the above-described Escherichia coli NM522/pQE60ywfE, which is a microorganism carrying a recombinant DNA comprising DNA having the nucleotide sequence shown in SEQ ID NO: 8.

(b) Acquisition of the Microorganism which Produces a Protein Having the Dipeptide-synthesizing Activity

The microorganism which produces a protein having the dipeptide-synthesizing activity can be obtained by introducing the DNA obtained by the method described in the above (a) into a host cell using the methods described in Molecular Cloning, Second Edition, Current Protocols in Molecular Biology, etc., for example, in the following manner.

On the basis of the DNA obtained by the method described in the above (a), a DNA fragment of an appropriate length comprising a region encoding the protein having the dipeptide-synthesizing activity is prepared according to need. The productivity of the protein can be enhanced by replacing a nucleotide in the nucleotide sequence of the region encoding the protein so as to make a codon most suitable for the expression in a host cell.

The DNA fragment is inserted downstream of a promoter in an appropriate expression vector to prepare a recombinant DNA.

A transformant producing the protein having the dipeptide-synthesizing activity can be obtained by introducing the recombinant DNA into a host cell suited for the expression vector.

As the host cell, any microorganisms that are capable of expressing the desired gene can be used.

The expression vectors that can be employed are those capable of autonomous replication or integration into the chromosome in the above host cells and comprising a promoter at a position appropriate for the transcription of the DNA encosing a protein having the dipeptide-synthesizing activity.

When a procaryote such as a bacterium is used as the host cell, it is preferred that the recombinant DNA comprising the DNA encoding a protein having the dipeptide-synthesizing activity is a recombinant DNA which is capable of autonomous replication in the procaryote and which comprises a promoter, a ribosome binding sequence, the DNA encoding a protein having the dipeptide-synthesizing activity, and a transcription termination sequence. The recombinant DNA may further comprise a gene regulating the promoter.

Examples of suitable expression vectors are pBTrp2, pBTac1 and pBTac2 (products of Boehringer Mannheim GmbH), pHelix1 (Roche Diagnostics Corp.), pKK233-2 (Amersham Pharmacia Biotech), pSE280 (Invitrogen Corp.), pGEMEX-1 (Promega Corp.), pQE-8 (Qiagen, Inc.), pET-3 (Novagen, Inc.), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (Takara Bio Inc.), pUC118 (Takara Bio Inc.) and pPA1 (Japanese Published Unexamined Patent Application No. 233798/88).

As the promoter, any promoters capable of functioning in host cells such as Escherichia coli can be used. For example, promoters derived from Escherichia coli or phage, such as trp promoter (Pₜᵣₚ), lac promoter (P_{lac}), P_{L} promoter, P_{R} promoter and P_{SE} promoter, SPO1 promoter, SPO2 promoter and penP promoter can be used. Artificially designed and modified promoters such as a promoter in which two Pₜᵣₚs are combined in tandem, tac promoter, lacT7 promoter and letI promoter, etc. can also be used.

Also useful are promoters such as xy1A promoter for the expression in bacteria belonging to the genus Bacillus [Appl. Microbiol. Biotechnol., 35, 594-599 (1991)] and P54-6 promoter for the expression in bacteria belonging to the genus Corynebacterium [Appl. Microbiol. Biotechnol., 53, 674-679 (2000)].

It is preferred to use a plasmid in which the distance between the Shine-Dalgarno sequence (ribosome binding sequence) and the initiation codon is adjusted to an appropriate length (e.g., 6 to 18 nucleotides).

In the recombinant DNA wherein the DNA encoding a protein having the dipeptide-synthesizing activity is ligated to an expression vector, the transcription termination sequence is not essential, but it is preferred to place the transcription termination sequence immediately downstream of the structural gene.

An example of such recombinant DNA is pPE43 described below.

Examples of procaryotes which are preferably used as host cells include microorganisms belonging to the genera Escherichia, Bacillus, Pseudomonas and Corynebacterium. Specific examples of microorganisms belonging to these genera are Escherichia coli XL1-Blue, Escherichia coli XL2-Blue, Escherichia coli DH1, Escherichia coli DH5α, Escherichia coli MC1000, Escherichia coli KY3276, Escherichia coli W1485, Escherichia coli JM101, Escherichia coli JM109, Escherichia coli HB101, Escherichia coli No. 49, Escherichia coli w3110, Escherichia coli NY49, Escherichia coli MP347, Escherichia coli NM522, Bacillus subtilis (ATCC 33712), Bacillus megaterium, Bacillus sp. (FERM BP-6030), Bacillus amyloliquefaciens, Bacillus coagulans, Bacillus licheniformis, Bacillus pumilus, Pseudomonas putida, Corynebacterium glutamicum (ATCC 13032) and Corynebacterium glutamicum (ATCC 14297).

Introduction of the recombinant DNA can be carried out by any of the methods for introducing DNA into the above host cells, for example, the method using calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

When a strain belonging to the genus Saccharomyces is used as the host cell, YEp13 (ATCC 37115), YEp24 (ATCC 37051), YCp50(ATCC 37419), pHS19, pHS15, etc. can be used as the expression vector.

As the promoter, any promoters capable of functioning in strains belonging to the genus Saccharomyces can be used. Suitable promoters include PHO5 promoter, PGK promoter, GAP promoter, ADR promoter, gal 1 promoter, gal 10 promoter, heat shock polypeptide promoter, MFα1 promoter and CUP 1 promoter.

Examples of suitable host cells are strains belonging to the genus Saccharomyces, specifically, Saccharomyces cerevisiae.

Introduction of the recombinant DNA can be carried out by any of the methods for introducing DNA into yeast, for example, electroporation [Methods Enzymol., 194, 182 (1990)], the spheroplast method [Proc. Natl. Acad. Sci. USA, 81, 4889 (1984)] and the lithium acetate method [J. Bacteriol., 153, 163 (1983)].

3. Production of the Enzyme Source Used in the Process for Producing a Dipeptide of the Present Invention

The enzyme source used in the process for producing a dipeptide of the present invention can be obtained by culturing the microorganism obtained in the above 2 in a natural or synthetic medium suitable for efficient culturing of the microorganism which contains carbon sources, nitrogen sources, inorganic salts, etc. which can be assimilated by the microorganism to obtain a culture of the microorganism or a treated matter of the culture, and then treating the culture or treated matter thereof at a temperature of 41 to 65° C. for 30 seconds to one hour (hereinafter also referred to simply as heat treatment).

As the carbon sources, any carbon sources that can be assimilated by the microorganism can be used. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

As the nitrogen sources, ammonia, ammonium salts of organic or inorganic acids such as ammonium chloride, ammonium sulfate, ammonium acetate and ammonium phosphate, and other nitrogen-containing compounds can be used as well as peptone, meat extract, yeast extract, corn steep liquor, casein hydrolyzate, soybean cake, soybean cake hydrolyzate, and various fermented microbial cells and digested products thereof.

Examples of the inorganic salts include potassium dihydrogenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration. The culturing temperature is preferably 15 to 40° C., and the culturing period is usually 5 hours to 7 days. The pH is maintained at 3.0 to 9.0 during the culturing. The pH adjustment is carried out by using an organic or inorganic acid, an alkali solution, urea, calcium carbonate, ammonia, etc.

If necessary, antibiotics such as ampicillin and tetracycline may be added to the medium during the culturing.

When a microorganism transformed with an expression vector comprising an inducible promoter is used in the process for producing a dipeptide of the present invention, an inducer may be added to the medium, if necessary. For example, in the case of a microorganism transformed with an expression vector comprising lac promoter, isopropyl-β-D-thiogalactopyranoside or the like may be added to the medium; and in the case of a microorganism transformed with an expression vector comprising trp promoter, indoleacrylic acid or the like may be added.

The enzyme source used in the production process of the present invention can be produced by applying heat treatment to the culture of the microorganism or treated matter thereof obtained above at a temperature of 41 to 65° C. for 30 seconds to one hour. The heat treatment may be carried out under any conditions within the range of from 41 to 65° C. for 30 seconds to one hour, preferably from 45 to 65° C. for one to 30 minutes, more preferably from 50 to 65° C. for 5 to 20 minutes, further preferably from 50 to 65° C. for 10 to 15 minutes, so long as it does not deprive the microorganism used as the enzyme source of the activity to produce a dipeptide and it can reduce the activity of the microorganism to decompose the dipeptide. Those skilled in the art can easily select heat treatment conditions suitable for the microorganism used in the process for producing a dipeptide of the present invention by simple experiments.

The heat treatment of the culture of the microorganism or treated matter thereof can be carried out in such a state that the culture or treated matter thereof is allowed to be present in an aqueous medium. An example of the aqueous medium is an aqueous medium used for the reaction to form a dipeptide described below. There is no restriction as to when the heat treatment is to be carried out so long as it is before contacting the substrate such as amino acids, etc. with the enzyme source. For example, in the case of using the treated matter of the culture of the microorganism as the enzyme source, the treated matter of the culture may be produced after giving heat treatment to the culture, or the treated matter of the culture may be produced prior to the heat treatment.

4. Process for Producing a Dipeptide of the Present Invention

The production process of the present invention contemplates a process for producing a dipeptide which comprises allowing an enzyme source and one or more kinds, preferably one or two kinds of substances selected from the group consisting of amino acid amides, amino acid esters and amino acids to be simultaneously present in an aqueous medium, said enzyme source being the culture of a microorganism or treated matter thereof subjected to heat treatment which is obtained in the above 3, allowing the dipeptide to form and accumulate in the medium and recovering the dipeptide from the medium.

Specifically, the production processes of the present invention include the following (i) to (iii): (i) a process for producing a dipeptide, which comprises: allowing an enzyme source and one or more kinds, preferably one or two kinds of amino acids to be present in an aqueous medium, said enzyme source being the culture of a microorganism or treated matter thereof subjected to heat treatment which is obtained in the above 3; allowing the dipeptide to form and accumulate in the medium; and recovering the dipeptide from the medium;
(ii) a process for producing a dipeptide, which comprises: allowing an enzyme source, one or more kinds, preferably one kind of amino acid ester and one or more kinds, preferably one kind of amino acid to be present in an aqueous medium, said enzyme source being the culture of a microorganism or treated matter thereof subjected to heat treatment which is obtained in the above 3; allowing the dipeptide to form and accumulate in the medium; and recovering the dipeptide from the medium; and
(iii) a process for producing a dipeptide, which comprises: allowing an enzyme source, one or more kinds, preferably one kind of amino acid amide and one or more kinds, preferably one kind of amino acid to be present in an aqueous medium, said enzyme source being the culture of a microorganism or treated matter thereof subjected to heat treatment which is obtained in the above 3; allowing the dipeptide to form and accumulate in the medium; and recovering the dipeptide from the medium.

One or more kinds, preferably one or two kinds of amino acids used as substrates in the above production process (i) are amino acids, preferably amino acids selected from the group consisting of L-amino acids, glycine (Gly) and β-alanine (β-Ala), and can be used in any combination. Examples of L-amino acids are L-alanine (L-Ala), L-glutamine (L-Gln), L-glutamic acid (L-Glu), L-valine (L-Val), L-leucine (L-Leu), L-isoleucine (L-Ile), L-proline (L-Pro), L-phenylalanine (L-Phe), L-tryptophan (L-Trp), L-methionine (L-Met), L-serine (L-Ser), L-threonine (L-Thr), L-cysteine (L-Cys), L-asparagine (L-Asn), L-tyrosine (L-Tyr), L-lysine (L-Lys), L-arginine (L-Arg), L-histidine (L-His), L-aspartic acid (L-Asp), L-α-aminobutyric acid (L-α-AB), L-azaserine, L-theanine, L-4-hydroxyproline (L-4-HYP), L-3-hydroxyproline (L-3-HYP), L-ornithine (L-Orn), L-citrulline (L-Cit) and L-6-diazo-5-oxo-norleucine.

The amino acids which are more preferably used in the above process (i) include the following: a combination of one kind of amino acid selected from the group consisting of L-Ala, Gly, L-Met, L-Ser, L-Thr and β-Ala, and one kind of amino acid selected from the group consisting of L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-azaserine, L-theanine, L-4-HYP, L-3-HYP, L-Orn, L-Cit and L-6-diazo-5-oxo-norleucine; a combination of L-Gln and L-Phe; and a combination of L-α-AB and L-Gln, L-Arg or L-α-AB. Further preferred amino acids are: a combination of L-Ala and one kind of amino acid selected from the group consisting of L-Ala, L-Gln, Gly, L-Val, L-Leu, L-Ile, L-Phe, L-Trp, L-Met, L-Ser, L-Thr, L-Cys, L-Asn, L-Tyr, L-Lys, L-Arg, L-His, L-α-AB, L-azaserine, L-Cit and L-theanine; a combination of Gly and one kind of amino acid selected from the group consisting of L-Gln, Gly, L-Phe, L-Trp, L-Met, L-Ser, L-Thr, L-Cys, L-Tyr, L-Lys, L-Arg L-α-AB and L-Cit; a combination of L-Met and one kind of amino acid selected from the group consisting of L-Phe, L-Met, L-Ser, L-Thr, L-Cys, L-Tyr, L-Lys and L-His; a combination of L-Ser and one kind of amino acid selected from the group consisting of L-Gln, L-Phe, L-Ser, L-Thr, L-Tyr, L-His and L-α-AB; a combination of L-Thr and one kind of amino acid selected from the group consisting of L-Gln, L-Phe, L-Leu, L-Thr and L-α-AB; a combination of L-Gln and L-Phe; a combination of β-Ala and one kind of amino acid selected from the group consisting of L-Phe, L-Met, L-His and L-Cit; and a combination of L-α-AB and L-Gln, L-Arg or L-α-AB.

In the above production process (i), the amino acid used as a substrate is added to the aqueous medium at the start or in the course of reaction to give a concentration of 0.1 to 500 g/l, preferably 0.2 to 200 g/l.

The dipeptides produced by the above process (i) include the dipeptides represented by the following formula (I):
R¹—R² (I)
(wherein R¹ and R², which may be the same or different, each representing an amino acid). Preferred dipeptides are those represented by the above formula (I) wherein R¹ and R², which may be the same or different, each represent an amino acid selected from the group consisting of L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-azaserine, L-theanine, L-4-HYP, L-3-HYP, L-Orn and L-6-diazo-5-oxo-norleucine. More preferred are dipeptides wherein R¹ is L-Ala, Gly, LI-Met, L-Ser, L-Thr or β-Ala, and R² is L-Ala, L-Gln, L-Glu, Gly, L-Val, L-Leu, L-Ile, L-Pro, L-Phe, L-Trp, L-Met, L-Ser, L-Thr, L-Cys, L-Asn, L-Tyr, L-Lys, L-Arg, L-His, L-Asp, L-α-AB, β-Ala, L-azaserine, L-theanine, L-4-HYP, L-3-HYP, L-Orn or L-6-diazo-5-oxo-norleucine Further preferred dipeptides are: dipeptides wherein R¹ is L-Ala and R² is L-Ala, L-Gln, Gly, L-Val, L-Leu, L-Ile, L-Phe, L-Trp, L-Met, L-Ser, L-Thr, L-Cys, L-Asn, L-Tyr, L-Lys, L-Arg, L-His, L-α-AB, L-azaserine or L-theanine; dipeptides wherein R¹ is Gly and R² is L-Gln, Gly, L-Trp, L-Met, L-Ser, L-Thr, L-Cys, L-Tyr, L-Lys, L-Arg or L-α-AB; dipeptides wherein R¹ is L-Met and R² is L-Phe, L-Met, L-Cys, L-Tyr, L-Lys or L-His; dipeptides wherein R¹ is L-Ser and R² is L-Gln, Gly, L-Phe, L-Met, L-Ser, L-Thr, L-Tyr, L-His or L-α-AB; dipeptides wherein R¹ is L-Thr and R² is L-Gln, L-Gly, L-Phe, L-Met, L-Ser, L-Thr or L-α-AB; dipeptides wherein R¹ is L-Gln and R² is L-Phe or L-α-AB; a dipeptide wherein R¹ is L-Phe and R² is L-Gln; a dipeptide wherein R¹ is L-Trp and R² is Gly; dipeptides wherein R¹ is L-Cys and R² is L-Ala, L-Gln, Gly, or L-Met; dipeptides wherein R¹ is L-Lys, and R² is L-Ala, Gly or L-Met; a dipeptide wherein R¹ is L-Arg and R² is L-α-AB; a dipeptide wherein R¹ is L-His and R² is L-Met; and dipeptides wherein R¹ is L-α-AB and R² is L-Ala, L-Gln, Gly, L-Ser, L-Thr, L-Arg or L-α-AB.

Further, in the above process, compounds which can be metabolized by the microorganism of the present invention to produce ATP, for example, sugars such as glucose, alcohols such as ethanol, and organic acids such as acetic acid may be added, as ATP source, to the aqueous medium according to need.

One or more kinds of amino acid esters and one or more kinds of amino acids used as substrates in the above production process (ii) may be any of amino acid esters and amino acids that can be used as substrates by the microorganism used as an enzyme source of the process of the present invention to form a dipeptide, and they can be used in any combination. Preferably, a combination of one kind of L-amino acid ester and one kind of amino acid is used, and it is preferred to use L-amino acid and Gly as the amino acid. More preferred combinations of one kind of amino acid ester and one kind of amino acid include combinations of one kind of amino acid ester selected from the group consisting of L-alanine ester, glycine ester, L-valine ester, L-isoleucine ester, L-methionine ester, L-phenylalanine ester, L-serine ester, L-threonine ester, L-glutamine ester, L-tyrosine ester, L-arginine ester, L-aspartic acid-α-ester, L-aspartic acid-β-ester, L-leucine ester, L-asparagine ester, L-lysine ester, L-aspartic acid-α, β-dimethyl ester and L-glutamine-γ-ester, and one kind of amino acid selected from the group consisting of L-Gln, L-Asn, Gly, L-Ala, L-Leu, L-Met, L-Pro, L-Phe, L-Trp, L-Ser, L-Thr, L-Tyr, L-Lys, L-Arg, L-His and L-Glu.

In the above process (ii), the amino acid ester and the amino acid used as substrates are added to the aqueous medium at the start or in the course of reaction to give a concentration of 0.1 to 500 g/l, preferably 0.2 to 200 g/l, respectively.

One or more kinds of amino acid amides and one or more kinds of amino acids used as substrates in the above production process (iii) may be any of amino acid amides and amino acids that can be used as substrates by the microorganism used as an enzyme source of the process of the present invention to form a dipeptide, and they can be used in any combination. Preferably, a combination of one kind of amino acid amide and one kind of amino acid is used, and it is preferred to use L-amino acid and Gly as the amino acid. Examples of combinations of one kind of amino acid amide and one kind of amino acid include combinations of one kind of amino acid amide selected from the group consisting of L-alanine amide, glycine amide and L-aspartic acid amide, and one kind of amino acid selected from the group consisting of L-Gln, L-Asn, Gly, L-Ala, L-Val, L-Leu, L-Ile, L-Met, L-Pro, L-Phe, L-Trp, L-Ser, L-Thr, L-Tyr, L-Lys, L-Arg, L-His and L-Glu.

In the above process (iii), the amino acid amide and the amino acid used as substrates are added to the aqueous medium at the start or in the course of reaction to give a concentration of 0.1 to 500 g/l, preferably 0.2 to 200 g/l, respectively.

The aqueous medium used in the production processes of the present invention may comprise any components and may have any composition so far as the dipeptide-forming reaction is not inhibited. Suitable aqueous media include water and buffers such as phosphate buffer, carbonate buffer, acetate buffer, borate buffer, citrate buffer and Tris buffer. The aqueous medium may comprise alcohols such as methanol and ethanol, esters such as ethyl acetate, ketones such as acetone, and amides such as acetamide.

The dipeptide-forming reaction is carried out in the aqueous medium at pH 5 to 11, preferably pH 6 to 10, at 20 to 60° C., preferably 25 to 45° C., for 2 to 150 hours, preferably 6 to 120 hours.

If necessary, a surfactant or an organic solvent may further be added to the aqueous medium.

Any surfactant that promotes the formation of a dipeptide can be used. Suitable surfactants include nonionic surfactants such as polyoxyethylene octadecylamine (e.g., Nymeen S-215, NOF Corporation), cationic surfactants such as cetyltrimethylammonium bromide and alkyldimethylbenzylammonium chloride (e.g., Cation F2-40E, NOF Corporation), anionic surfactants such as lauroyl sarcosinate, and tertiary amines such as alkyldimethylamine (e.g., Tertiary Amine FB, NOF Corporation), which may be used alone or in combination. The surfactant is usually used at a concentration of 0.1 to 50 g/l. As the organic solvent, xylene, toluene, aliphatic alcohols, acetone, ethyl acetate, etc. may be used usually at a concentration of 0.1 to 50 ml/l.

The treated matters of the culture include the treated matter comprising living cells such as concentrated culture, dried culture, cells obtained by centrifuging the culture, and products obtained by treating the cells by various means such as drying, freeze-drying, treatment with a surfactant, treatment with a solvent, enzymatic treatment and immobilization. The treated matters of the culture of the present invention also include crude extracts of protein obtained by removing insoluble matters and the like from the treated matters obtained by treating the above cells by means such as treatment with ultrasonication and mechanical friction.

The amount of the culture or a treated matter of the culture used as the enzyme source to be added varies according to its specific activity, etc., but is, for example, 5 to 1000 mg, preferably 10 to 400 mg per mg of the amino acid, amino acid ester or amino acid amide used as a substrate.

Recovery of the dipeptide formed and accumulated in the aqueous medium can be carried out by ordinary methods using active carbon, ion-exchange resins, etc. or by means such as extraction with an organic solvent, crystallization, thin layer chromatography and high performance liquid chromatography.

Further, the above production processes (ii) and (iii) can be carried out according to the descriptions in WO03/010189 or WO03/010187.

5. Experimental Examples of the Process for Producing a Microorganism Which Produces a Dipeptide from One or More Kinds of Amino Acids

Experimental examples of the process for producing a microorganism which produces a dipeptide from one or more kinds of amino acids are shown below.

### EXPERIMENTAL EXAMPLE 1

Construction of a Plasmid Expressing ywfE Gene Derived from Bacillus Subtilis

A ywfE gene fragment of Bacillus subtilis was obtained in the following manner.

By using a DNA synthesizer (Model 8905, PerSeptive Biosystems, Inc.), DNAs having the nucleotide sequences shown in SEQ ID NOS: 25 and 26 (hereinafter referred to as primer A and primer B, respectively) were synthesized. Primer A has a nucleotide sequence containing a region wherein the initiation codon of ywfE gene (atg) is substituted by the NcoI recognition sequence (ccatgg). Primer B has a nucleotide sequence containing a region wherein the termination codon of ywfE gene is substituted by the BamHI recognition sequence (ggatcc).

PCR was carried out using the chromosomal DNA of Bacillus subtilis as a template and the above primer A and primer B as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 u mol/l each of the primers, 2.5 units of Pfu DNA polymerase, 4 μl of buffer for Pfu DNA polymerase (10×) and 200 μmol/l each of dNTPs.

One-tenth of the resulting reaction mixture was subjected to agarose gel electrophoresis to confirm that a ca. 1.4 kb fragment corresponding to the ywfE gene fragment was amplified. Then, the remaining reaction mixture was mixed with an equal amount of phenyl/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained solution (5 μl) was subjected to reaction to cleave the amplified DNA with restriction enzymes NcoI and BamBI. DNA fragments were separated by agarose gel electrophoresis, and a 1.4 kb DNA fragment containing ywfE gene was recovered using GENECLEAN II Kit(BIO 101).

C-Terminal His-tagged recombinant expression vector pQE60 (Qiagen, Inc.) (0.2 μg) was cleaved with restriction enzymes NcoI and BamHI. DNA fragments were separated by agarose gel electrophoresis, and a 3.4 kb DNA fragment was recovered in the same manner as above.

The 1.4 kb DNA fragment containing ywfE gene and the 3.4 kb DNA fragment obtained above were subjected to ligation reaction using a ligation kit (Takara Bio Inc.) at 16° C. for 16 hours.

Escherichia coli NM522 (Stratagene) was transformed using the ligation reaction mixture according to the method using calcium ion [Proc. Natl. Acad. Sci. USA, 69, 2110 (1972)], spread on LB agar medium containing 50 μg/ml ampicillin, and cultured overnight at 30° C.

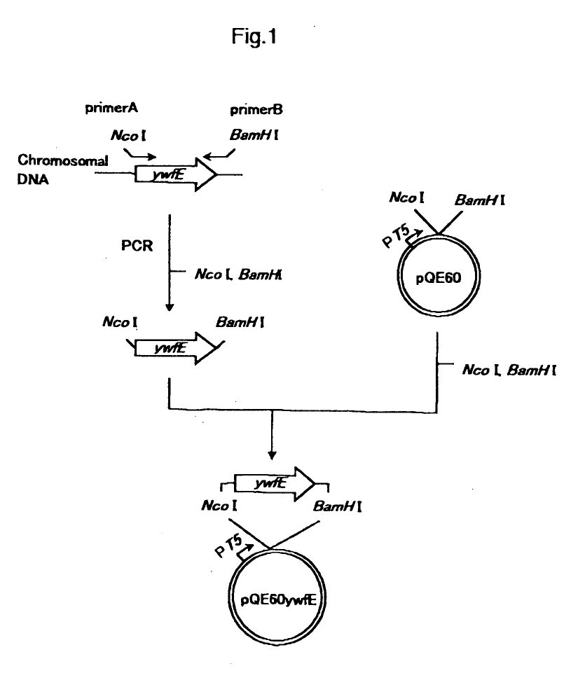

A plasmid was extracted from a colony of the transformant that grew on the medium according to a known method, whereby pQE60ywfE, which is a C-terminal His-tagged type of ywfE gene expression vector, was obtained. The structure of the vector was confirmed by digestion with restriction enzymes (FIG. 1).

### EXPERIMENTAL EXAMPLE 2

Acquisition of a ywfE Gene Product

Escherichia coli NM522/pQE60ywfE carrying pQE60ywfE was inoculated into 8 ml of LB medium containing 50 μg/ml ampicillin in a test tube, and cultured at 28° C. for 17 hours. The resulting culture was inoculated into 50 ml of LB medium containing 50 μg/ml ampicillin in a 250-ml Erlenmeyer flask, and cultured at 30° C. for 3 hours. Then, isopropyl-β-D-thiogalactopyranoside (IPTG) was added to give a final concentration of 1 mmol/l, followed by further culturing at 30° C. for 4 hours. The resulting culture was centrifuged to obtain wet cells, and a His-tagged recombinant enzyme was purified from the wet cells using HisTrap (His-tagged protein purification kit, Amersham Pharmacia Biotech) according to the instructions attached thereto.

### EXPERIMENTAL EXAMPLE 3

Production of Dipeptides Using the His-Tagged Recombinant Enzyme (1)

(i) A reaction mixture (0.1 ml) comprising 0.04 mg of the purified His-tagged recombinant enzyme obtained in Experimental Example 2, 100 mmol/l Tris-HCl (pH 8.0), 60 mmol/l magnesium chloride, 60 mmol/l ATP, 30 mmol/l L-Ala and 30 mmol/l L-Gln was prepared, and reaction was carried out at 37° C. for 16 hours.

After the completion of reaction, the reaction product was derivatized by the dinitrophenyl method and then analyzed by HPLC. The HPLC analysis was carried out using, as a separation column, Lichrosorb-RP-18 column (Kanto Kagaku) and, as an eluting solution, 1% (v/v) phosphoric acid and 25% (v/v) acetonitrile at a flow rate of 0.7 ml/min. As a result, it was confirmed that 3.7 g/l L-Ala-L-Gln and 0.3 g/l L-alanyl-L-alanine (L-Ala-L-Ala) were formed and accumulated in the reaction mixture.
(ii) Reactions were carried out under the same conditions as in the above (i) using reaction mixtures having the same composition as that of the reaction mixture of the above (i) except that 0.01 mg of the enzyme was used and L-Phe, L-Met, L-Leu and L-Val, respectively, were used in place of L-Gln.

After the completion of reactions, the reaction products were analyzed in the same manner as in the above (i), whereby it was confirmed that the following dipeptides were formed and accumulated in the respective reaction mixtures: 7.0 g/l L-alanyl-L-phenylalanine (L-Ala-L-Phe) alone; 7.0 g/l L-alanyl-L-methionine (L-Ala-L-Met) and 0.03 g/l L-Ala-L-Ala; 5.0 g/l L-alanyl-L-leucine (L-Ala-L-Leu) and 0.2 g/l L-Ala-L-Ala; and 1.6 g/l L-alanyl-L-valine (L-Ala-L-Val) and 0.3 g/l L-Ala-L-Ala.
(iii) Reactions were carried out under the same conditions as in the above (i) using reaction mixtures having the same composition as that of the reaction mixture of the above (i) except that 0.01 mg of the enzyme was used, Gly was used in place of L-Ala, and L-Phe and L-Met, respectively, were used in place of L-Gln.

After the completion of reactions, the reaction products were analyzed in the same manner as in the above (i), whereby it was confirmed that 5.2 g/l glycyl-L-phenylalanine (Gly-L-Phe) and 1.1 g/l glycyl-L-methionine (Gly-L-Met) were formed and accumulated in the respective reaction mixtures.

When ATP was excluded from the compositions of the above reaction mixtures, no dipeptide was formed.

The above results revealed that the ywfE gene product has the activity to produce, in the presence of ATP, the following dipeptides: L-Ala-L-Gln plus L-Ala-L-Ala, L-Ala-L-Phe, L-Ala-L-Met plus L-Ala-L-Ala, L-Ala-L-Leu plus L-Ala-L-Ala, or L, Ala-L-Val plus L-Ala-L-Ala from L-Ala plus L-Gln, L-Phe, L-Met, L-Leu or L-Val; and Gly-L-Phe or Gly-L-Met from Gly plus L-Phe or L-Met.

### EXPERIMENTAL EXAMPLE 4

Production of Dipeptides Using the His-Tagged Recombinant Enzyme (2)

A reaction mixture (0.1 ml) comprising 0.04 mg of the purified His-tagged recombinant enzyme obtained in Experimental Example 2, 100 mmol/l Tris-HCl (pH 8.0), 60 mmol/l magnesium chloride and 60 mmol/l ATP was prepared. To this mixture were respectively added combinations of various L-amino acids, Gly and i-Ala selected from the amino acids shown in the first row of Table 1 and in the leftmost column of Table 1 to give a concentration of 30 mmol/l each, and the resulting mixtures were subjected to reaction at 37° C. for 16 hours. After the completion of reactions, the reaction products were analyzed by HPLC, whereby it was confirmed that the dipeptides shown in Table 1 were formed.

TABLE 1

| | Ala | Gln | Glu | Gly | Val | Leu | Ile | Pro | Phe | Trp | Met | Ser |
|---|---|---|---|---|---|---|---|---|---|---|---|---|
| Ala | AlaAla | AlaGln | AlaAla | AlaGly | AlaVal | AlaLeu | AlaIle | AlaAla | AlaPhe | AlaTrp | AlaMet | AlaSer |
| | | AlaAla | | AlaAla | AlaAla | AlaAla | AlaAla | | AlaAla | AlaAla | | AlaAla |
| Gln | | X | X | GlyGln | X | X | X | X | ◯ | X | MetMet | SerGln |
| | | | | GlyGly | | | | | | | | SerSer |
| Glu | | | | GlyGly | | | | | | | | |
| Gly | | | | GlyGly | | | | GlyGly | GlyPhe | GlyGly | GlyMet | GlySer |
| | | | | | | | | | | ◯ | GlyGly | GlyGly |
| | | | | | | | | | | | | SerGly |
| | | | | | | | | | | | | SerSer |
| Val | | | | | | | | | | | X | |
| Leu | | | | | | | | | | | MetMet | |
| Ile | | | | | | | | | | | MetMet | |
| Pro | | | | | | | | | | | MetMet | SerSer |
| Phe | | | | | | | | | | | MetPhe | SerPhe |
| | | | | | | | | | | | MetMet | |
| Trp | | | | | | | | | | | | |
| Met | | | | | | | | | | | MetMet | SerMet |
| Ser | | | | | | | | | | | | SerSer |
| Thr | | | | | | | | | | | | |
| Cys | | | | | | | | | | | | |
| Asn | | | | | | | | | | | | |
| Tyr | | | | | | | | | | | | |
| Lys | | | | | | | | | | | | |
| Arg | | | | | | | | | | | | |
| His | | | | | | | | | | | | |
| Asp | | | | | | | | | | | | |
| α-AB | | | | | | | | | | | | |
| β-Ala | | | | | | | | | | | | |
| Cit | | | | | | | | | | | | |

| | | | | | | | | | | | | A [symbol] - | Th [symbol] - |
|---|---|---|---|---|---|---|---|---|---|---|---|---|---|
| | Thr | Cys | Asn | Tyr | Lys | Arg | His | Asp | α-AB | β-Ala | Cit | serine | nine |
| Ala | AlaThr | AlaAla | AlaAsn | AlaTyr | AlaAla | AlaArg | AlaHis | AlaAla | AlaAla | | AlaAla | AlaAla | AlaAla |
| | AlaAla | ◯ | AlaAla | AlaAla | ◯ | AlaAla | AlaAla | | ◯ | | ◯ | ◯ | ◯ |
| Gln | ThrGln | ◯ | X | X | X | X | X | X | ◯ | | | | |
| | ThrThr | | | | | | | | | | | | |
| Glu | | | | | | | | | | | | | |
| Gly | GlyThr | GlyGly | GlyGly | GlyTyr | GlyGly | GlyArg | GlyGly | GlyGly | GlyGly | | ◯ | | |
| | GlyGly | ◯ | | GlyGly | ◯ | GlyGly | | | ◯ | | | | |
| | ThrGly | | | | | | | | | | | | |
| | ThrThr | | | | | | | | | | | | |
| Val | | | | | | | | | | | | | |
| Leu | ThrLeu | | | | | | | | | | | | |
| Ile | | | | | | | | | | | | | |
| Pro | ThrThr | | | | | | | | | | | | |
| Phe | ThrPhe | | | | | | | X | | ◯ | | | |
| | ThrThr | | | | | | | | | | | | |
| Trp | | | | | | | | | | | | | |
| Met | ThrMet | MetMet | | MetTyr | MetMet | | MetMet | | | ◯ | | | |
| | ThrThr | ◯ | | MetMet | ◯ | | ◯ | | | | | | |
| Ser | SerThr | | | SerTyr | | | SerHis | | SerSer | | | | |
| | SerSer | | | SerSer | | | | | ◯ | | | | |
| | ThrSer | | | | | | | | | | | | |
| | ThrThr | | | | | | | | | | | | |
| Thr | ThrThr | | | | | | | | ThrThr | | | | |
| | | | | | | | | | ◯ | | | | |
| Cys | | | | | | | | | | | | | |
| Asn | | | | | | | | | | | | | |
| Tyr | | | | | | | | | | | | | |
| Lys | | | | | | | | | | | | | |
| Arg | | | | | | | | | ◯ | | | | |
| His | | | | | | | | | | β-AlaHis | | | |
| Asp | | | | | | | | | | | | | |
| α-AB | | | | | | | | | ◯ | | | | |
| β-Ala | | | | | | | | | | | | | |
| Cit | | | | | | | | | | ◯ | | | |

The dipeptides formed by the reaction using, as substrates, two (or one) kinds of L-amino acids, Gly and β-Ala shown in the first row and the leftmost column of Table 1 are shown in the respective cells of the table. In the table, ◯ means that a dipeptide was formed though its sequence was unidentified; x means that formation of a dipeptide was not confirmed; and a blank means that reaction was not carried out.

### EXPERIMENTAL EXAMPLE 5

Production of a Dipeptide Using the Strain Expressing the His-Tagged Recombinant Enzyme

Escherichia coli NM522/pQE60ywfE obtained in Experimental Example 1 was inoculated into 8 ml of LB medium containing 50 μg/ml ampicillin in a test tube, and cultured at 28° C. for 17 hours The resulting culture was inoculated into 50 ml of LB medium containing 50 μg/ml ampicillin in a 250-ml Erlenmeyer flask, and cultured at 30° C. for 3 hours. Then, IPTG was added to give a final concentration of 1 mmol/l, followed by further culturing at 30° C. for 4 hours. The resulting culture was centrifuged to obtain wet cells.

A reaction mixture (20 ml, pH 7.2) comprising 200 g/l wet cells, 50 g/l glucose, 5 g/l phytic acid (diluted to neutrality with 33% conc. sodium hydroxide solution), 15 g/l potassium dihydrogenphosphate, 5 g/l magnesium sulfate heptahydrate, 4 g/l Nymeen S-215, 10 ml/l xylene, 200 mmol/l L-Ala and 200 mmol/l L-Gln was put in a 50-ml beaker, and reaction was carried out at 32° C. at 900 rpm for 2 hours During the reaction, the pH of the reaction mixture was maintained at 7.2 by using 2 mol/l potassium hydroxide.

The reaction product was analyzed by the same method as in Experimental Example 3, whereby it was confirmed that 25 mg/l L-Ala-L-Gln was accumulated.

### EXPERIMENTAL EXAMPLE 6

Cloning of Genes Corresponding to the ywfE Gene from Various Microorganisms of the Genus Bacillus and Analysis Thereof

On the basis of the nucleotide sequence shown in SEQ ID NO: 8, genes corresponding to the ywfE gene which exist in Bacillus subtilis ATCC 15245, ATCC 6633, IAM 1213, IAM 1107, IAM 1214, ATCC 9466, IAM 1033 and ATCC 21555, Bacillus amyloliquefaciens IFO 3022 and Bacillus pumilus NRRL B-12025 were obtained in the following manner.

That is, Bacillus subtilis ATCC 15245, ATCC 6633, IAM 1213, IAM 1107, IAM 1214, ATCC 9466, IAM 1033 and ATCC 21555, Bacillus amyloliquefaciens IFO 3022 and Bacillus pumilus NRRL B-12025 were respectively inoculated into LB medium and subjected to static culture overnight at 30° C. After the culturing, the chromosomal DNAs of the respective microorganisms were isolated and purified according to the method using saturated phenyl described in Current Protocols in Molecular Biology.

By using a DNA synthesizer (Model 8905, PerSeptive Biosystems, Inc.), DNAs having the nucleotide sequences shown in SEQ ID NOS: 27 and 28 (hereinafter referred to as primer C and primer D, respectively) were synthesized. Primer C has a sequence containing a region upstream of the initiation codon of ywfE gene of the chromosomal DNA of Bacillus subtilis 168, and primer D has a sequence complementary to a sequence containing a region downstream of the termination codon of ywfE gene.

PCR was carried out using each of the chromosomal DNAs of Bacillus subtilis ATCC 15245, ATCC 6633, IAM 1213, IAM 1107, IAM 1214, ATCC 9466, IAM 1033 and ATCC 21555 and Bacillus amyloliquefaciens IFO 3022 as a template and the above primer C and primer D as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 mmol/l each of dNTPs.

One-tenth of each of the resulting reaction mixtures was subjected to agarose gel electrophoresis to confirm that a ca. 1.4 kb fragment corresponding to the ywfE gene fragment was amplified. Then, the remaining reaction mixture was mixed with an equal amount of phenyl/chloroform saturated with TE. The resulting solution was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

Each of the thus obtained 1.4 kb DNA fragments derived from the chromosomal DNAs of the respective strains and pCR-blunt (Invitrogen Corp.) were subjected to ligation reaction using a ligation kit at 16° C. for 16 hours.

Escherichia coli NH522 was transformed using each ligation reaction mixture according to the method using calcium ion, spread on LB agar medium containing 50 μg/ml ampicillin, and cultured overnight at 30° C.

A plasmid was extracted from a colony of each transformant that grew on the medium according to a known method and the structure of each plasmid was analyzed using restriction enzymes. As a result, it was confirmed that the following plasmids containing a gene corresponding to the ywfE gene were obtained: pYWFE1 (derived from ATCC 15245, DNA having the nucleotide sequence shown in SEQ ID NO: 30), pYwFE2 (derived from ATCC 6633, DNA having the nucleotide sequence shown in SEQ ID NO: 9), pYWFE3 (derived from IAM 1213, DNA having the nucleotide sequence shown in SEQ ID NO: 10), pYWFE4 (derived from IAM 1107, DNA having the nucleotide sequence shown in SEQ ID NO: 11), pYWFE5 (derived from IAM 1214, DNA having the nucleotide sequence shown in SEQ ID NO: 12), pYWFE6 (derived from ATCC 9466, DNA having the nucleotide sequence shown in SEQ ID NO: 8), pYWFE7 (derived from IAM 1033, DNA having the nucleotide sequence shown in SEQ ID NO: 30), pYWFE8 (derived from ATCC 21555, DNA having the nucleotide sequence shown in SEQ ID NO: 13) and pYWFE9 (derived from IFO 3022, DNA having the nucleotide sequence shown in SEQ ID NO: 14).

On the other hand, a gene corresponding to ywfE gene derived from Bacillus pumilus NRRL B-12025 (DNA having the nucleotide sequence shown in SEQ ID NO: 29) was obtained in the following manner.

PCR was carried out using the chromosomal DNA of the NRRL B-12025 strain prepared above as a template and DNAs respectively consisting of the nucleotide sequences shown in SEQ ID NOS: 31 and 32 as a set of primers. That is, PCR was carried out by 30 cycles, one cycle consisting of reaction at 98° C. for 5 seconds, reaction at 55° C. for 30 seconds and reaction at 72° C. for one minute, using 50 μl of a reaction mixture comprising 0.1 μg of the chromosomal DNA, 0.5 mmol/l each of the primers, 2.5 units of Z-taq polymerase (Takara Bio Inc.), 5 μl of buffer for Z-taq polymerase (10×) (Takara Bio Inc.) and 200 μmol/l each of dNTPs.

One-tenth of the resulting reaction mixture was subjected to agarose gel electrophoresis to confirm that a ca. 0.8 kb fragment was amplified. Then, the remaining reaction mixture was mixed with an equal amount of phenyl/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained 0.8 kb fragment derived from the chromosomal DNA and pGEM T-easy (Promega Corp.) were subjected to ligation reaction using a ligation kit at 16° C. for 16 hours.

Escherichia coli DR α was transformed using the reaction mixture according to the method using calcium ion, spread on LB agar medium containing 50 μg/ml ampicillin, and cultured overnight at 30° C.

A plasmid was extracted from the transformant obtained above and the nucleotide sequence of the ca. 0.8 kb DNA insert was determined, whereby a sequence from nucleotides 358 to 1160 in the nucleotide sequence shown in SEQ ID NO: 29 was confirmed.

The above plasmid was cleaved with EcoRI and then subjected to agarose gel electrophoresis to separate a DNA fragment. The DNA fragment was purified using GENECLEAN II Kit, and about 0.5 μg of the purified DNA fragment was DIG-labeled using DIG-High Prime DNA Labeling & Detection Starter Kit I (Roche Diagnostics Corp.) according to the instructions attached thereto.

Southern analysis of the chromosomal DNA of the NRRL B-12025 strain was carried out using the DIG-labeled DNA obtained above.

The chromosomal DNA of the NRRL B-12025 strain was completely digested with BamHI, EcoRI, HindIII, KpnI, PstI, SacI, SalI and SphI, respectively, and subjected to agarose gel electrophoresis to separate DNA fragments, followed by transfer to nylon membrane plus charge (Roche Diagnostics Corp.) according to an ordinary method.

After the DNA fragments were fixed on the nylon membrane by UV irradiation, Southern hybridization was carried out using the above probe DNA and the nylon membrane.

The hybridization was carried out by contacting the nylon membrane with the probe DNA at 65° C. for 16 hours, washing the nylon membrane twice with a solution consisting of 0.1% SDS and 2×SSC at room temperature for 5 minutes, and further washing the membrane twice with a solution consisting of 0.1% SDS and 0.5×SSC at 65° C. for 15 minutes. The other operations and conditions and detection of the hybridized DNA were carried out according to the instructions attached to the above-mentioned DIG-High Prime DNA Labeling & Detection Starter Kit I.

As a result, color development was observed at around 3.5 kbp of the fragments completely digested with HindIII and PstI.

Subsequently, the chromosomal DNA of the NRRL B-12025 strain was completely digested with HindIII and PstI, respectively, and subjected to agarose gel electrophoresis to separate DNA fragments. From the respective restriction enzyme-digested DNAs, 3-4 kbp fragments were purified using GENECLEAN II Kit, followed by autocyclization using the ligation kit.

On the basis of the nucleotide sequence of the 0.8 kb DNA fragment determined above, the nucleotide sequences shown in SEQ ID NOS: 33 and 34 were designed and synthesized, and they were used in PCR using the cyclized DNA obtained above as a template. PCR was carried out by 30 cycles, one cycle consisting of reaction at 98° C. for 5 seconds, reaction at 55° C. for 30 seconds and reaction at 72° C. for 3 minutes and 30 seconds, using 50 μl of a reaction mixture comprising 10 ng of the cyclized DNA, 0.5 mmol/1 each of the primers, 2.5 units of pyrobest polymerase (Takara Bio Inc.), 5 μl of buffer for pyrobest polymerase (10×) (Takara Bio Inc.) and 200 μmol/l each of dNTPs.

One-tenth of the resulting reaction mixture was subjected to agarose gel electrophoresis to confirm that a ca. 3.0 kb fragment was amplified. Then, the remaining reaction mixture was mixed with an equal amount of phenyl/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

The thus obtained DNA fragment and Zero Blunt PCR Cloning Kit (Invitrogen Corp.) were subjected to ligation reaction using a ligation kit.

Escherichia coli NM522 was transformed using the reaction mixture according to the method using calcium ion, spread on LB agar medium containing 50 μg/ml ampicillin, and cultured overnight at 30° C.

A plasmid was extracted from a colony of the transformant that grew on the medium according to a known method and the structure of the plasmid was analyzed using restriction enzymes. As a result, it was confirmed that plasmid pYWFE10 (derived from NRRL B-12025, DNA having the nucleotide sequence shown in SEQ ID NO: 29) containing a gene corresponding to the ywfE gene was obtained.

The nucleotide sequences of the genes corresponding to the ywfE gene which are respectively contained in the plasmids pYWFE1 to pYWFE10 obtained above were determined using 373A DNA Sequencer.

The amino acid sequences of the proteins encoded by the genes respectively contained in pYWFE1, pYWFE6 and pYWFE7 were identical with the amino acid sequence of the protein encoded by the ywfE gene, whereas those of the proteins encoded by the genes respectively contained in pYWFE2, pYWFE3, pYWFE4, pYWFE5, pYWFE8, pYWFE9 and pYWFE10 were different from the amino acid sequence of the protein encoded by the ywfE gene.

The amino acid sequences of the proteins encoded by the genes corresponding to the ywfE gene which are contained in pYWFE2, pYWFE3, pYWFE4, pYWFE5, pYWFE8, pYWFE9 and pYWFE10, and pYWFE1 and pYWFE7 are shown in SEQ ID NOS: 2 to 7, 35 and 1, respectively, and the nucleotide sequences of these genes are shown in SEQ ID NOS; 9 to 14, 29, 8 and 30, respectively.

### EXPERIMENTAL EXAMPLE 7

Purification of C-Terminal His-Tagged Recombinant Dipeptide Synthetase

PCR was carried out using each of the chromosomal DNAs of Bacillus subtilis ATCC 15245, ATCC 6633, IAM 1213, IAM 1107, IAM 1214, ATCC 9466, IAM 1033 and ATCC 21555 and Bacillus amyloliquefaciens IFO 3022 as a template and primer A and primer B described in Experimental Example 1 as a set of primers. That is, PCR was carried out by 30 cycles, one cycle consisting of reaction at 94° C. for one minute, reaction at 55° C. for 2 minutes and reaction at 72° C. for 3 minutes, using 40 μl of a reaction mixture comprising 0.1 μg of the chromosomal DNA, 0.5 μmol/l each of the primers, 2.5 units of Pfu DNA polymerase, 4 μl of buffer for Pfu DNA polymerase (10×) and 200 μmol/l each of dNTPs.

When the chromosomal DNA of Bacillus pumilus NRRL B-12025 was used as a template, PCR was carried out using DNAs respectively having the nucleotide sequences shown in SEQ ID NOS: 36 and 37 as a set of primers under the same conditions as above.

One-tenth of each of the resulting reaction mixtures was subjected to agarose gel electrophoresis to confirm that a ca. 1.4 kb DNA fragment corresponding to the ywfE fragment was amplified. Then, the remaining reaction mixture was mixed with an equal amount of phenyl/chloroform saturated with TE. The resulting mixture was centrifuged, and the obtained upper layer was mixed with a two-fold volume of cold ethanol and allowed to stand at −80° C. for 30 minutes. The resulting solution was centrifuged, and the obtained DNA precipitate was dissolved in 20 μl of TE.

Each of the thus obtained solutions (5 μl) was subjected to reaction to cleave the amplified DNA with restriction enzymes NcoI and BamHI. DNA fragments were separated by agarose gel electrophoresis, and a 1.4 kb DNA fragment containing a gene corresponding to the ywfE gene was recovered using GENECLEAN II Kit.

Subsequently, 0.2 μg of the C-terminal His-tagged recombinant expression vector pQE60 was cleaved with restriction enzymes NcoI and BamHI. DNA fragments were separated by agarose gel electrophoresis, and a 3.4 kb DNA fragment was recovered in the same manner as above.

Each of the 1.4 kb DNA fragments containing a gene corresponding to the ywfE gene of Bacillus subtilis 168 and the 3.4 kb DNA fragment obtained above were subjected to ligation reaction using a ligation kit at 16° C. for 16 hours. Escherichia coli NM522 was transformed using each ligation reaction mixture according to the method using calcium ion, spread on LB agar medium containing 50 μg/ml ampicillin, and cultured overnight at 30° C.

A plasmid was extracted from a colony of each transformant that grew on the medium according to a known method and the structure of each plasmid was analyzed using restriction enzymes. As a result, it was confirmed that the following C-terminal His-tagged gene expression vectors were obtained: pQE60ywfE1 (a vector containing the gene derived from ATCC 15245), pQE60ywfE2 (a vector containing the gene derived from ATCC 6633), pQE60ywfE3 (a vector containing the gene derived from IAM 1213), pQE60ywfE4 (a vector containing the gene derived from IAM 1107), pQE60ywfE5 (a vector containing the gene derived from IAM 1214), pQE60ywfE6 (a vector containing the gene derived from ATCC 9466), pQE60ywfE7 (a vector containing the gene derived from IAM 1033), pQE60ywfE8 (a vector containing the gene derived from ATCC 21555), pQE60ywfE9 (a vector containing the gene derived from IFO 3022) and pQE60ywfE10 (a vector containing the gene derived from NRRL B-12025).

Escherichia coli NM522/pQE60ywfE1 to NM522/pQE60ywfE10 strains obtained above were respectively inoculated into 8 ml of LB medium containing 50 μg/ml ampicillin in a test tube, and cultured at 28° C. for 17 hours. Each of the resulting cultures was inoculated into 50 ml of LB medium containing 50 μg/ml ampicillin in a 250-ml Erlenmeyer flask, and cultured at 30° C. for 3 hours. Then, IPTG was added to give a final concentration of 1 mmol/l, followed by further culturing at 30° C. for 4 hours. The resulting culture was centrifuged to obtain wet cells, and His-tagged recombinant enzymes were purified from the respective wet cells using HisTrap according to the instructions attached thereto.

### EXPERIMENTAL EXAMPLE 8

Production of Dipeptides using Purified Enzymes

Reaction mixtures (0.1 ml each) comprising 0.04 mg of the respective recombinant enzymes obtained in Experimental Example 7, 100 mmol/l Tris-HCl (pH 8.0), 60 mmol/l magnesium chloride, 60 mmol/l ATP, 30 mmol/l L-Ala and 30 mmol/l L-Gln were prepared, and reactions were carried out at 37° C. for 16 hours.

After the completion of reactions, the reaction mixtures were analyzed by the method described in Experimental Example 3, whereby it was confirmed that 3.0 to 3.5 g/l L-Ala-L-Gln and 0.25 to 0.3 g/l L-Ala-L-Ala were formed and accumulated.

When ATP was excluded from the compositions of the above reaction mixtures, L-Ala-L-Gln or L-Ala-L-Ala was not formed at all.

The above results revealed that all of the products of the genes obtained in Experimental Example 7 have the activity to produce L-Ala-L-Gln and L-Ala-L-Ala from L-Ala and L-Gln in the presence of ATP.

The present invention is illustrated more in detail in the following examples. These examples are not to be construed as limiting the scope of the invention.

### EXAMPLE 1

Production of L-Alanyl-L-Glutamine (L-Ala-L-Gln)

A liquid medium (30 ml, pH 7.0) containing 5 g/l glucose, 5 g/l ammonium sulfate, 1 g/l potassium dihydrogenphosphate, 3 g/l dipotassium hydrogenphosphate, 0.5 g/l magnesium sulfate, 10 g/l yeast extract and 10 g/l peptone was put in a 300-ml Erlenmeyer flask and sterilized at 115° C. for 15 minutes. Corynebacterium glutamicum ATCC 13286 was cultured at 30° C. for 24 hours using a slant agar medium having the same composition as that of the liquid medium (containing 20 g/l agar, pH 7.0), and one platinum loop of the cultured cells was inoculated into the liquid medium and cultured with shaking at 120 reciprocation/min. at 30° C. for 17 hours. After the completion of culturing, the culture was centrifuged, and 100 mmol/l borate buffer (pH 9.0) was added to the resulting wet cells to give a concentration of 100 g/l. One-ml aliquots of the thus prepared cell suspension were subjected to: 1) no heat treatment; 2) heat treatment at 50° C. for 10 minutes; or 3) heat treatment at 550C for 10 minutes. The cell suspensions were then added, respectively, to one ml of a reaction solution [20 mmol/l EDTA, 200 mmol/l L-alanine ethyl ester hydrochloride, 400 mmol/l L-glutamine, 100 mmol/l borate buffer, pH 9.0] to bring total volume to 2 ml, followed by incubation at 30° C. for 30 minutes. The reaction mixture was heated at 90° C. for 15 minutes and then centrifuged, and the amount of L-Ala-L-Gln in the resulting supernatant was determined by HPLC.

HPLC analysis was carried out after the reaction product was derivatized by the dinitrophenyl method using, as a separation column, Lichrosorb-RP-18 column (Kanto Kagaku) and, as an eluting solution, a solution comprising 1% (v/v) phosphoric acid and 25% (v/v) acetonitrile at a flow rate of 0.7 ml/min. The results are shown in Table 2.

TABLE 2

| | Amount of L-Ala-L-Gln |
|---|---|
| Heat treatment conditions | formed (relative value) |
| No heat treatment | 100 |
| At 50° C. for 10 min. | 353 |
| At 55° C. for 10 min. | 329 |

The amount of L-Ala-L-Gln formed was increased to more than 3-fold by carrying out heat treatment.

### EXAMPLE 2

Activity of Dipeptide-Producing Strain to Decompose L-Ala-L-Gln

Corynebacterium glutamicum ATCC 13286 was cultured and a cell suspension was obtained in the same manner as in Example 1. One-ml aliquots of the cell suspension were subjected to: 1) no heat treatment; 2) heat treatment at 50° C. for 15 minutes; or 3) heat treatment at 65° C. for 15 minutes. The cell suspensions were then added, respectively, to one ml of a reaction solution [20 mmol/l EDTA, 2 g/l L-Ala-L-Gln, 100 mmol/l borate buffer, pH 9.0] to bring total volume to 2 ml, followed by incubation at 30° C. for 30 minutes. The reaction mixture was heated at 90° C. for 15 minutes and then centrifuged, and the amount of L-Ala-L-Gln in the resulting supernatant was determined by HPLC in the same manner as in Example 1.

As a result, the amount of L-Ala-L-Gln decomposed by the 30-minute reaction was, when no heat treatment is designated as 100, 84 for the heat treatment at 55° C. for 15 minutes and 50 for the heat treatment at 65° C. for 15 minutes.

The above results show that the amount of L-Ala-L-Gln formed was increased because the activity to decompose L-Ala-L-Gln was suppressed by the heat treatment.

### EXAMPLE 3

Activity of Non-Dipeptide-Producing Strain to Decompose L-Ala-L-Gln

Escherichia coli JM109 was cultured and a cell suspension was obtained in the same manner as in Example 1. One-ml aliquots of the cell suspension were subjected to: 1) no heat treatment; 2) heat treatment at 55° C. for 15 minutes; or 3) heat treatment at 65° C. for 15 minutes. The cell suspensions were then added, respectively, to one ml of a reaction solution [20 mmol/l EDTA, 2 g/l L-Ala-L-Gln, 100 mmol/l borate buffer, pH 9.0] to bring total volume to 2 ml, followed by incubation at 300C for 30 minutes. The reaction mixture was heated at 90° C. for 15 minutes and then centrifuged, and the amount of L-Ala-L-Gln in the resulting supernatant was determined by HPLC in the same manner as in Example 1.

As a result, the amount of L-Ala-L-Gln decomposed by the 30-minute reaction was, when no heat treatment is designated as 100, 0 for the heat treatment at 55° C. for 15 minutes and 7 for the heat treatment at 65° C. for 15 minutes.

The results of Examples 1 and 2 and the above results revealed that the amount of a dipeptide formed is remarkably improved by using, as an enzyme source, a heat-treated product of a culture of a microorganism transformed with DNA encoding a protein concerned with formation of a dipeptide, for example, DNA encoding a protein consisting of the amino acid sequence shown in any of SEQ ID NOS: 1 to 7, 17 to 19 and 23, thereby imparted with the ability to produce a dipeptide, or that of a treated matter of the culture.

SEQUENCE LISTING FREE TEXT

| | |
|---|---|
| SEQ ID NO: 25 | Description of Artificial Sequence: |
| | Synthetic DNA |
| SEQ ID NO: 26 | Description of Artificial Sequence: |
| | Synthetic DNA |
| SEQ ID NO: 27 | Description of Artificial Sequence: |
| | Synthetic DNA |
| SEQ ID NO: 28 | Description of Artificial Sequence: |
| | Synthetic DNA |
| SEQ ID NO: 31 | Description of Artificial Sequence: |
| | Synthetic DNA |
| SEQ ID NO: 32 | Description of Artificial Sequence: |
| | Synthetic DNA |
| SEQ ID NO: 33 | Description of Artificial Sequence: |
| | Synthetic DNA |
| SEQ ID NO: 34 | Description of Artificial Sequence: |
| | Synthetic DNA |
| SEQ ID NO: 36 | Description of Artificial Sequence: |
| | Synthetic DNA |
| SEQ ID NO: 37 | Description of Artificial Sequence: |
| | Synthetic DNA |

1. A process for producing a dipeptide, which comprises:
allowing an enzyme source and one or more kinds of substances selected from the group consisting of amino acid amides, amino acid esters and amino acids to be present in an aqueous medium, said enzyme source being a culture of a microorganism having the ability to produce a dipeptide or a treated matter of the culture and having been subjected to heat treatment at a temperature of 41 to 65° C. for 30 seconds to one hour so as to produce and accumulate the dipeptide in the aqueous medium;
allowing the dipeptide to form and accumulate in the aqueous medium; and
recovering the dipeptide from the aqueous medium.

2. The process according to claim 1, wherein the microorganism having the ability to produce a dipeptide is a microorganism belonging to the genus Achromobacter, Acinetobacter, Aeromonas, Agrobacterium, Alcaligenes, Arthrobacter, Beijerinckia, Brevibacterium, Clavibacter, Chryseobacterium, Escherichia, Enterobacter, Erwinia, Flavobacterium, Kluyvera, Microbacterium, Micrococcus, Mycoplana, Pantoea, Propionibacterium, Listonella, Rhizobium, Rhodococcus, Salmonella, Sarcina, Serratia, Staphylococcus, Stenotrophomonas, Streptomyces, Vibrio, Xanthomonas, Bullera, Candida, Cryptococcus, Filobasidium, Geotrichum, Pachysolen, Rhodosporidium, Rhodotorula, Saccharomyces, Sporobolomyces, Tremella, Torulaspora, Torulopsis, Gluconacetobacter, Acetobacter, Gluconobacter, Asaia, Zucharibacter, Actinomadura, Kitasatosporia, Micromonospora, Nocardia, Oerskovia, Saccharothrix, Streptoverticillium, Hafnia, Lactobacillus, Neisseria, Thermoplasma, Corynebacterium, Pseudomonas, Bacillus, Trichosporon or Sterigmatomyces.

3. The process according to claim 1, wherein the microorganism having the ability to produce a dipeptide is a microorganism having the ability to produce a protein according to any of [1] to [4] below:
[1] a protein having the amino acid sequence shown in any of SEQ ID NOS: 1 to 7 and 35;
[2] a protein consisting of an amino acid sequence wherein one or more amino acid residues are deleted, substituted or added in the amino acid sequence shown in any of SEQ ID NOS: 1 to 7 and 35 and having the activity to synthesize a dipeptide;
[3] a protein consisting of an amino acid sequence which has 65% or more homology to the amino acid sequence shown in any of SEQ ID NOS: 1 to 7 and 35 and having the activity to synthesize a dipeptide; and
[4] a protein comprising an amino acid sequence which has 80% or more homology to the amino acid sequence shown in SEQ ID NO: 15 and having the activity to synthesize a dipeptide.

4. The process according to claim 1, wherein the microorganism having the ability to produce a dipeptide is a microorganism carrying DNA according to any of [1] to [4] below:
[1] DNA encoding the protein according to any of [1] to [4] of claim 3; [2] DNA having the nucleotide sequence shown in any of SEQ ID NOS: 8 to 14, 29 and 30;
[3] DNA which hybridizes with DNA having the nucleotide sequence shown in any of SEQ ID NOS: 8 to 14, 29 and 30 under stringent conditions and which encodes a protein having the activity to synthesize a dipeptide; and
[4] DNA comprising a nucleotide sequence which has 80% or more homology to the nucleotide sequence shown in SEQ ID NO: 16 and encoding a protein having the activity to synthesize a dipeptide.

5. The process according to claim 1, wherein the microorganism having the ability to produce a dipeptide is a microorganism producing a protein having proline iminopeptidase activity or a protein having L-amino acid amide hydrolase activity.

6. The process according to claim 5, wherein the protein having proline iminopeptidase activity is a protein according to any of [1] to [3] below:
[1] a protein having the amino acid sequence shown in any of SEQ ID NOS: 17 to 19;
[2] a protein consisting of an amino acid sequence wherein one or more amino acid residues are deleted, substituted or added in the amino acid sequence shown in any of SEQ ID NOS: 17 to 19 and having proline iminopeptidase activity; and
[3] a protein consisting of an amino acid sequence which has 80% or more homology to the amino acid sequence shown in any of SEQ ID NOS: 17 to 19 and having proline iminopeptidase activity.

7. The process according to claim 5, wherein the microorganism producing a protein having proline iminopeptidase activity is a microorganism carrying DNA according to any of [1] to [3] below:
[1] DNA encoding the protein according to any of [1] to [3] of claim 6;
[2] DNA having the nucleotide sequence shown in any of SEQ ID NOS: 20 to 22; and
[3] DNA which hybridizes with DNA having the nucleotide sequence shown in any of SEQ ID NOS: 20 to 22 under stringent conditions and which encodes a protein having proline iminopeptidase activity.

8. The process according to claim 5, wherein the protein having L-amino acid amide hydrolase activity is a protein according to any of [1] to [3] below:
[1] a protein having the amino acid sequence shown in SEQ ID NO: 23;
[2] a protein consisting of an amino acid sequence wherein one or more amino acid residues are deleted, substituted or added in the amino acid sequence shown in SEQ ID NO: 23 and having L-amino acid amide hydrolase activity; and
[3] a protein consisting of an amino acid sequence which has 80% or more homology to the amino acid sequence shown in SEQ ID NO: 23 and having L-amino acid amide hydrolase activity.

9. The process according to claim 5, wherein the microorganism producing a protein having L-amino acid amide hydrolase activity is a microorganism carrying DNA according to any of [1] to [3] below:
[1] DNA encoding the protein according to any of [1] to [3] of claim 8;
[2] DNA having the nucleotide sequence shown in SEQ ID NO: 24; and
[3] DNA which hybridizes with DNA having the nucleotide sequence shown in SEQ ID NO: 24 under stringent conditions and which encodes a protein having L-amino acid amide hydrolase activity.

10. The process according to claim 1, wherein the microorganism having the ability to produce a dipeptide is a microorganism carrying a recombinant DNA in which the DNA according to any of [1] to [4] of claim 4, [1] to [3] of claim 7 and [1] to [3] of claim 9 is ligated to a vector DNA.

11. The process according to claim 10, wherein the microorganism carrying a recombinant DNA is a microorganism belonging to the genus Escherichia, Bacillus, Pseudomonas, Corynebacterium or Saccharomyces.

12. The process according to claim 1, wherein the amino acid is an amino acid selected from L-amino acids, glycine and β-alanine.

13. The process according to claim 12, wherein the L-amino acid is one or more kinds of amino acids selected from the group consisting of L-alanine, L-glutamine, L-glutamic acid, L-valine, L-leucine, L-isoleucine, L-proline, L-phenylalanine, L-tryptophan, L-methionine, L-serine, L-threonine, L-cysteine, L-asparagine, L-tyrosine, L-lysine, L-arginine, L-histidine, L-aspartic acid, L-α-aminobutyric acid, L-azaserine, L-theanine, L-4-hydroxyproline, L-3-hydroxyproline, L-ornithine and L-6-diazo-5-oxo-norleucine.

14. The process according to claim 1, wherein the amino acid ester is one or more kinds of amino acid esters selected from the group consisting of L-alanine ester, glycine ester, L-valine ester, L-isoleucine ester, L-methionine ester, L-phenylalanine ester, L-serine ester, L-threonine ester, L-glutamine ester, L-tyrosine ester, L-arginine ester, L-aspartic acid-α-ester, L-aspartic acid-α-ester, L-leucine ester, L-asparagine ester, L-lysine ester, L-aspartic acid-α,β-dimethyl ester and L-glutamine-γ-ester, and the amino acid is one or more kinds of amino acids selected from the group consisting of L-glutamic acid, L-glutamine, L-asparagine, glycine, L-alanine, L-leucine, L-methionine, L-proline, L-phenylalanine, L-tryptophan, L-serine, L-threonine, L-tyrosine, L-lysine, L-arginine and L-histidine.

15. The process according to claim 1, wherein the amino acid amide is one or more kinds of amino acid amides selected from the group consisting of L-alanine amide, glycine amide and L-aspartic acid-α-amide, and the amino acid is one or more kinds of amino acids selected from the group consisting of L-glutamic acid, L-asparagine, L-glutamine, glycine, L-alanine, L-valine, L-leucine, L-isoleucine, L-methionine, L-proline, L-phenylalanine, L-tryptophan, L-serine, L-threonine, L-tyrosine, L-lysine, L-arginine and L-histidine.

16. The process according to claim 1, wherein the treated matter of the culture is concentrated culture, dried culture, cells obtained by centrifuging the culture, or a product obtained by subjecting the cells to drying, freeze-drying, treatment with a surfactant, treatment with a solvent, enzymatic treatment or immobilization.


SEQUENCE LISTING (37 sequences)

SEQ ID NO: 1 - protein, 472 aa, Bacillus subtilis 168
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVSFSAAAPGTSVDLTLFEAFNSIAAFELKGSNSQDVAESIRQIQQHAKLTAKYVLPV

SEQ ID NO: 2 - protein, 472 aa, Bacillus subtilis ATCC6633
MERKTVLVIADLGGCPPHMFYKSAAEKYNLVSFIPRPFAITASHAALIEKYSVAVIKDKDYFQSLADFEHPDSIYWAHEDHDKPEEEVVEQIVKVAQMFEADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIVTFSAAAPGTSVDLTLFEAFNSIAAFELKGSNSQDVAESIRQIQQHAKLTAKYVLPV

SEQ ID NO: 3 - protein, 472 aa, Bacillus subtilis IAM1213
MERKTVLVIADLGGCPPHMFYKSAAEKYNLVSFIPRPFAITASHAALIEKYSVAVIKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEIKLMKNREPGLIESAARFAGWNMIPNIKKVFGLDMAQLLLDVLCFGKDADLPDGLLDQEPYYVADCHLYPQHFKQNGQIPETAEDLVIEAIDLPDGLLKGDTEIVSFSAAAPGTSVDLTLFEAFNSIAAFELKGSNSQDVAESIRQIQQHAKLTAKYVLPV

SEQ ID NO: 4 - protein, 472 aa, Bacillus subtilis IAM1107
MERKTVLVIADLGGCPPHMFYKSAAEKYNLVSFIPRPFAITASHAALIEKYSVAVVKDKDYFKSLADFEHPDSIYWAHEDHNKPEEEVVEQIVKVAEMFGADAITTNNELFIAPMAKACERLGLRGAGVQAAENARDKNKMRDAFNKAGVKSIKNKRVTTLEDFRAALEEIGTPLILKPTYLASSIGVTLITDTETAEDEFNRVNDYLKSINVPKAVTFEAPFIAEEFLQGEYGDWYQTEGYSDYISIEGIMADGEYFPIAIHDKTPQIGFTETSHITPSILDEEAKKKIVEAAKKANEGLGLQNCATHTEVKLMKNREPGLIESAARFAGWNMIPNIKKVFGLDMAQLLLDVLCFGKDADLPDGLLDQEPYYVADCHLYPQHFKQNGQIPETAEDLVIEAIDIPDGLLKGDTEIFSFSAAAPGTSVDLTLFEAFNSIAAFELKGSNSQDVAESIRQIQQHAKLTAKYVLPV

SEQ ID NO: 5 - protein, 472 aa, Bacillus subtilis IAM1214; identical to SEQ ID NO: 2

SEQ ID NO: 6 - protein, 472 aa, Bacillus subtilis ATCC21555
MERKTVLVIADLGGCPPHMFYKSAAEKYNLVSFIPRPFAITASHAALIEKYSIAVIKDKDYFKSLADFEHPDSIYWAHEDHDKPEEEVVEEIVKVADMFGVDAITTNNELFIAPMAKACKRLGLRGAGVQAAENARDKNKMRAAFNRAGVKSIKNKRVTTLEDFRAALQEIGTPLILKPTYLASSIGVTLIKEMETAEAEFNRVNEYLKSINVPKAVTFEAPFIAEEFLQGEYDDWYETSGYSDYISIEGIMADGEYFPVAIHDKTPQIGFTETAHITPSILDDDAKRKIVEAAKKANEGLGLENCATHTEIKLMKNREAGLIESAARFAGWNMIPNIKKVFGVDMAQLLLDVLCYGKEADLPKGLLEQEPCYVADCHLYPQHFKENGQLPETVVDFVIESIEIPDGVLKGDTELVSFSAAEAGTSVDLRLFEAFNSIAAFELKGSNSNDVAESIKQIQQQAKLTAKYALSV

SEQ ID NO: 7 - protein, 472 aa, Bacillus amyloliquefaciens IFO3022
MERKTVLVIADLGGCPPHMFYKSAAEKYNLVSFIPRPFAITASHAALIEKYSVAVIKDKDYFKSLADFEHPDSIYWAHEDHDKPEEEVVEEIVKVAGMFAVDAITTNNELFIAPMAKACERLGLRGAGVQAAENARDKNKMRAAFNRAGVKSIKNRRVTTLEDFRAALQEIGTPLILKPTYLASSIGVTLIKERETAEAEFNRVNEYLKSINVPKAVTFEAPFIAEEFLQGEYDDWYETSGYSDYISIEGIMADGEYFPVAIHDKTPQIGFTETSHITPSILDDDAKRKIVEAAKKANEGLGLENCATHTEIKLMKNREAGLIESAARFAGWNMIPNIKKVFGVDMAQLLLDVLCFGKEADLPKGLLEQEPCYVADCHLYPQHFKENGQLPETAVDFVIESIDIPDGVLKGDTEIVSFSAAEAGTSVDLRLFEAFNSIAAFELKGSNSGDVAESIKQIQQQAKLTAKYALPV

SEQ ID NO: 8 - DNA, 1416 bp, Bacillus subtilis 168
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgaacaccctgattccatttattgggcgcatgaagatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggactgcaaaattgcgcaacacatacagagatcaagctaatgaaaaacagagaaccgggtttaatagagtcggcagccagatttgccggctggaatatgatccccaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccaaattcctgaaactgctgaggatttggtcattgaagcgatcgatattccggacgggcttttaaaaggggatactgaaatcgtttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 9 - DNA, 1416 bp, Bacillus subtilis ATCC6633
atggagagaaaaacagtattggtcatcgctgatcttggaggctgcccgccgcacatgttttataaaagcgctgctgaaaaatataacctggttagctttattccgagaccttttgcaataacagcctcccatgcagcactgattgaaaaatactcggtcgcggtcataaaagataaagactattttcagagcttagctgattttgagcatcccgattcaatttattgggcgcatgaggatcatgacaagcctgaagaagaggttgtcgagcaaatcgtcaaggttgcccaaatgtttgaggcggacgccatcacaacaaacaatgaattattcattgccccgatggcgaaagcctgtgaacgccttggcctgaggggcgccggagtgcaggcagcggaaaatgccagagataaaaataaaatgagggacgcttttaataaggcgggagtcaaatcgatcaaaaacaaacgagtcacaactcttgaggattttcgtgctgcacttgaagagatcggcacacctctaatcttaaagcctacatacttagcgagttcaatcggcgtaacgctgattaccgacacggagacggcagaagatgaatttaacagagtcaatgactacctgaaatcgattaacgtgccgaaggcggtcacatttgaagcaccgtttattgctgaggaatttttacagggtgagtacggagactggtatcaaacagaagggtactccgactatatcagcatagaaggcattatggcagatggtgagtattttccgatcgccattcatgacaaaacgccgcaaattggatttacagagacatcacatattacgccatccattctggatgaagaggcgaaaaagaaaattgtcgaagcggctaaaaaggcaaatgaagggcttggactgcaaaattgcgcaacacatacagaaatcaagctaatgaaaaacagagaaccgggtttaatagagtcggctgccagattcgcaggctggaatatgattcctaacattaaaaaggttttcggccttgatatggcgcaattattattagatgttctctgtttcggaaaagatgctgatctgccggacgggttattggatcaagagccttactatgttgctgactgccatctgtaccctcagcatttcaaacaaaatggccagatccctgaaactgccgaggatttggtaatcgaagcgatcgatattccggatgggcttttgaagggtgatacagaaatcgttactttttcggctgcggcaccaggaacatcagttgatttgacactgtttgaagccttcaactccattgctgcatttgaactgaaaggcagcaattcacaggatgtggctgaatcaatcagacaaattcagcagcatgcgaagctgacggcaaagtatgtgctgccagta

SEQ ID NO: 10 - DNA, 1416 bp, Bacillus subtilis IAM1213
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgactccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactctcgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaaggtcttggcctgcaaaattgcgcaacacatacagagatcaagctaatgaaaaatagagaaccgggtttaattgaatcggcagccagattcgccggctggaatatgatccccaatattaaaaaggtctttggccttgatatggcgcaattattattagatgtcctttgtttcggaaaagacgccgatctgccggacggattattggatcaagagccttattatgttgccgactgccatttgtacccgcaacatttcaaacaaaatggccagattccagaaactgctgaggatttggtcattgaagcgatcgatctgcctgacgggcttttaaaaggggatactgagatcgtttctttttcggccgcagcaccaggaacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 11 - DNA, 1416 bp, Bacillus subtilis IAM1107
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcgtaaaagataaagactattttaagagtttagctgattttgagcatcctgactccatttattgggcgcatgaggatcataacaagcctgaggaagaggtcgtcgagcaaatcgtcaaggttgccgaaatgttcggggcggatgccatcacaacaaacaatgaattattcattgctccgatggcgaaagcctgtgaacgtctgggcttgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagaggtcaagctaatgaaaaacagagaaccgggtttaattgaatcggcagccagatttgccggctggaatatgatccctaacattaaaaaggttttcggccttgatatggcgcaattattattagatgtcctctgtttcggaaaagatgccgatctgccggacggattattggatcaagagccttactatgtcgccgactgccatttgtacccgcagcatttcaaacaaaatggccagattccagaaaccgctgaggatttggtcattgaagcgatcgatattccggacgggcttttaaaaggggatactgaaatcttttctttttcggccgcagcaccaggcacttcagttgatttgacattgtttgaagctttcaattccattgctgcatttgaactgaaaggcagtaattcacaggatgtggctgaatcaatcagacaaattcagcagcatgcgaagctgacggcaaagtatgtgctgccagta

SEQ ID NO: 12 - DNA, 1416 bp, Bacillus subtilis IAM1214; identical to SEQ ID NO: 9

SEQ ID NO: 13 - DNA, 1416 bp, Bacillus subtilis ATCC21555
atggagagaaaaacagtattggttatcgctgatcttgggggctgcccgccgcatatgttttacaaaagcgcagccgaaaaatacaacctcgtcagctttattccgagaccctttgcaattacagcctctcatgcggccttaattgaaaaatactcgattgcggtcattaaagataaagactattttaagagtctggctgattttgaacatcccgattcgatttattgggctcatgaagatcatgacaaacctgaggaagaagtcgtcgaagaaatcgtgaaagtggccgacatgtttggggttgacgccattacgaccaacaatgaactgtttatcgctccgatggcaaaagcgtgtaaacgtctcggcctgcggggagcgggcgtacaggccgctgaaaacgccagagataaaaataaaatgagagccgccttcaaccgggccggcgtcaaatccatcaaaaacaaacgggtgacgaccctggaagatttccgcgccgcgcttcaggaaatcggaacgccgcttattctgaagcctacatatctggcaagctcgatcggcgtgacgcttattaaagagatggaaacggccgaagctgaattcaacagagtcaatgagtacttgaaatcgattaatgtaccgaaagcggtgacgtttgaagcgccgtttatcgcggaagaattcttgcagggcgagtatgatgactggtacgaaacaagcggttattccgactatatcagcatcgaaggcatcatggccgacggagaatacttccccgttgcgatccatgataaaacaccgcaaatcggattcacggagacagcgcatattacgccgtccatcctggatgatgacgccaagcggaaaatcgtcgaagctgccaagaaggcgaatgaaggactcggcctcgaaaactgtgcaacgcatacagaaataaaattaatgaaaaaccgggaagccggactgattgagtcagcggccagattcgcgggatggaatatgattccgaatattaaaaaggtcttcggcgttgatatggcgcagctattattggatgttctctgttacggaaaagaagctgatctgccgaaaggattattggagcaggagccatgctatgtcgcagactgccacttgtatcctcagcatttcaaagagaacggccagctgcctgagacggttgtcgatttcgtcattgaaagcattgaaattcctgacggcgtcttaaagggagacactgaactcgtttctttctcagcggctgaggcgggtacgtcagtggatctgcggctgttcgaagcgttcaacagcattgcggcgtttgagctgaaaggaagcaattcgaacgacgtggccgaatcaatcaaacaaattcagcagcaggcgaagctgactgcaaagtatgcgttatcggta

SEQ ID NO: 14 - DNA, 1416 bp, Bacillus amyloliquefaciens IFO3022
atggagagaaaaacagtattggttatcgctgaccttgggggatgcccgccgcatatgttttacaaaagcgcagccgaaaaatacaacctcgtcagctttattccgagaccttttgcaattacagcctctcatgcggcattaattgaaaaatactcggtcgcggtcataaaagataaagactattttaagagtctggctgattttgagcatcccgattcgatttactgggctcatgaagatcatgacaaacctgaggaagaagtagtcgaagaaatcgtcaaggtggccggcatgttcgcggttgacgccattacgaccaacaatgaactgtttatcgctccgatggcaaaagcgtgtgaacgtctcggcctgcggggagcgggcgtacaggccgctgaaaatgccagagataaaaacaaaatgagagccgctttcaaccgggccggcgtcaagtctatcaaaaacagacgggtgacgacgctggaagatttccgcgccgcgcttcaggaaatcggaacgccgctcattctgaagcctacatatctggcgagctccatcggcgtgacgctcatcaaagagagggaaacggccgaagccgaatttaacagagtcaatgaatacctgaagtcgatcaacgtaccgaaagcggtcacgtttgaagcgccgtttatcgcggaagaatttttgcagggcgagtatgacgactggtacgaaacaagcggttattccgactatatcagcatagaaggcatcatggccgacggagaatacttccctgtcgcaattcatgataaaacaccgcaaatcggattcacggagacatcgcatattacgccgtccatcctggatgatgacgcgaagcggaaaatcgtcgaagcagccaaaaaggcgaatgaaggactcggcctcgaaaactgcgcaacccatacagagattaaattaatgaaaaaccgggaagccggactgattgaatcagcggcacgatttgcgggctggaacatgattccgaatattaaaaaggtcttcggcgtcgatatggcgcagctgttattggatgttctctgtttcggaaaagaagccgatctgccgaaaggattattggagcaggagccgtgctatgtcgccgactgccacttgtatcctcagcatttcaaagagaacggccagctgcctgagacggctgtcgatttcgtcattgaaagcattgacattcccgacggcgtcttaaagggagacaccgaaatcgtttctttctcggcggccgaggcgggtacatccgtggatctgcggctgttcgaagcgttcaacagcattgcggcgttcgagctgaaaggaagcaattcgggtgacgtggccgaatcaatcaaacaaattcagcagcaggcgaagctgactgcaaagtatgcgttaccggta

SEQ ID NO: 15 - protein, 93 aa, Bacillus subtilis 168
GAGVQAAENARDKNKMRDAFNKAGVKSIKNKRVTTLEDFRAALEEIGTPLILKPTYLASSIGVTLITDTETAEDEFNRVNDYLKSINVPKAVT

SEQ ID NO: 16 - DNA, 279 bp, Bacillus subtilis 168
ggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttctatcggtgtaacgctgattacggacactgagacggcagaagatgaatttaacagagtcaatgactatctgaaatcaattaacgtgccaaaggcggttacg

SEQ ID NO: 17 - protein, 413 aa, Corynebacterium glutamicum
  UNSURE (359): Other information: Xaa = unknown or other
  UNSURE (401): Other information: Xaa = unknown or other
MTKTLGSLQLEEITLTLPLTEDVADERTIDVFARIATRVGGEDLPYLVFLQGGPGNEAPRPSLNPLNPNWLGVALEEYRVVMLDQRGTGRSTPVGNDILEKPTAEVVEYLSHLRADGIVRDAEALRKHLGVNQWNLLGQSFGGFTTLHYLSRHADSLDNVFITGGLSAIDRPAEDVYANCYNRMRRNSEEFYRRFPQLRETFRGLVNRARAGEIVLPTGEVVSETRLRSLGHLLGSNDGWFDLYNLLELDPTSNAFVHDLAGLLPFGNRNPIYYVLHESSYADGVVTNWAAERVLPEDFREDPTLLTGEHVFQEWTDTVPSLKPWKDVALALAQQEWPKLYDAKALENSQAKGAAAVYXNDVFVPVDYSLETAQHLPGVQLFITSQHEHNGLRASSGAVLXHLFDLAHGREVR

SEQ ID NO: 18 - protein, 337 aa, Pseudomonas putida
MMPNGSQYPHTECAMQTLYPQIKPYARHDLAVEAPHVLYVDESGSPEGLPVVFIHGGPGAGCDAQSRCYFDPNLYRIITFDQRGCGRSTPHASLENNTTWHLVEDLERIREHLGIDKWVLFGGSWGSTLALAYAQTHPERVHGLILRGIFLCRPQEIEWFYQEGASRLFPDYWQDYIAPIPPEERGDLVKAFHKRLTGNDQIAQMHAAKAWSTWEGRTATLRPNPLVVDRFSEPQRALSIARIECHYFMNNAFLEPDQLIRDLPKIAHLPAVIVHGRYDVICPLDNAWALHQAWPNSELKVIRDAGHAASEPGITDALVRAADQMARRLLDLPLEEA

SEQ ID NO: 19 - protein, 323 aa, Pseudomonas putida
MQTLYPQIKPYVRHDLAVDETHTLYVDESGSPQGLPVVFIHGGPGAGCDANSRCYFDPNLYRIVTFDQRGCGRSTPRASLENNTTWDLVADLERIREHLGIEKWVLFGGSWGSTLALAYAQTHPDRVLGLIVRGIFLARPQDIQWFYQAGASRLFPDYWQDYIAPIPAEERHDMISAYHKRLTGNDQIAQMHAAKAWSTWEGRMLGLCPSPQLIERFSEPQRALSIARIECHYFTNNSFLEPNQLIRDMHKIAHLPGIIVHGRYDMICPLDNAWELHQAWPNSELQVIREAGHAASEPGITDALVRAAGDMARRLLDLPPEEA

SEQ ID NO: 20 - DNA, 1239 bp, Corynebacterium glutamicum
  UNSURE (359): Other information: Xaa = unknown or other
  UNSURE (401): Other information: Xaa = unknown or other
atgactaaaacacttggttcccttcaacttgaagaaattaccttgacgctccctctgactgaagatgtggccgatgaacgcaccattgatgtgttcgcacgcattgccacacgcgtcggtggggaagaccttccatatttagtattcctgcagggtgggcctggcaatgaagctccacgtccaagccttaatcccctcaaccccaattggttgggcgtggccttggaggaataccgcgtggtcatgttggatcaacgtggcaccggccgttccaccccagtgggtaatgatattttggaaaaacccacagcagaagtagtggagtacttatcccacctgcgcgcagatggcattgtgcgagatgctgaagccctgcgtaagcatttgggtgtgaatcagtggaaccttttaggccagtccttcggaggtttcaccaccctgcattacttgtcccggcacgccgattccttggacaacgtgtttattaccggcggtctcagcgctattgatcgcccagcagaagacgtgtatgccaactgttacaaccgcatgcgccgaaactctgaggaattctaccgtcgcttcccgcaattacgggaaactttccgagggttggttaatcgtgctcgcgccggggagattgtgcttcccaccggcgaagttgtgtcagaaaccaggctgcgatcccttggtcacttgttgggtagcaatgacggctggtttgatctgtacaacctgctggaattagatcccacctccaacgcttttgtccatgacctggcaggacttttgcctttcggcaaccgcaacccaatttattacgtgctccatgagtcctcttacgccgacggtgtggtgacaaattgggcagcagagcgtgtgcttccagaggatttccgcgaggatccaacactgctcaccggtgagcacgtgttccaggagtggacagacaccgtgccgtcgctcaagccgtggaaggacgttgccctggcattggctcagcaggaatggcccaagctttatgatgcgaaggcattggaaaactcacaggccaagggcgctgcagcagtgtatghcaatgacgttttcgtcccagtggattactctctggaaaccgcacaacacctgcccggtgtgcagctgtttatcaccagccagcatgaacacaatggacttcgtgccagctcaggcgcagtactgragcaccttttcgatctggcccacggccgagaggtacgc

SEQ ID NO: 21 - DNA, 1011 bp, Pseudomonas putida
atgatgcccaacggcagtcaatatcctcacacggagtgcgcaatgcagaccctctacccgcagatcaaaccctacgcccggcacgatctggccgtggaagcgccgcatgtgctgtatgtcgatgaaagcggctcgccggaaggtctgcccgtggtattcatccacggtggcccgggtgctggctgcgacgcccagagccgttgctactttgaccccaacctgtaccgcatcatcaccttcgaccagcgcggctgtggccgctccacgccccatgccagcctggagaacaacacaacctggcacctggtcgaggacctggagcgcatccgcgagcacctgggcatcgacaaatgggtgctgttcggcggctcgtggggttcgaccctggccctggcctacgcccagacccaccccgagcgtgtgcatgggctgatcctgcggggcatcttcctgtgccggccgcaggagatcgagtggttctaccaggagggcgccagccgcctgttccccgactactggcaggactacatcgcgccgattccgccggaagaacgcggcgacctggtcaaggccttccacaagcgcctcaccggtaacgatcagattgcccagatgcacgccgccaaggcgtggtctacctgggagggccgtaccgccaccctgcgcccgaacccgctggtggtcgaccgcttctccgagccgcagcgggcgctgtcgatcgcccggatcgagtgccactacttcatgaacaacgccttcctcgaaccggaccagttgatccgcgacctgccgaagatcgcccacctgccagcggtgatcgtgcacggtcgctatgacgtgatctgtccgctggacaacgcctgggccctgcaccaggcctggccgaacagcgaactgaaggtgatccgcgacgccggccacgccgcgtccgagccgggcatcaccgatgccctggtgcgggcagccgaccagatggcccggcgcctgctcgacctgcccctggaagaagca

SEQ ID NO: 22 - DNA, 969 bp, Pseudomonas putida
atgcagactttgtacccgcagatcaaaccctacgtccggcacgatctggccgtcgatgaaacccacacgctgtatgtcgacgaaagtggttccccgcaaggtttgcccgtggtcttcatccatggcggtcccggcgccggctgcgatgccaatagccgctgctatttcgatccgaacctgtaccgcatcgtcacctttgaccagcgcggctgcgggcgctccactccgcgggccagcctggaaaacaacaccacctgggacctggttgccgaccttgagcgcattcgcgagcacctggggattgaaaaatgggtgctgttcggtggttcctggggctcgaccctggccctggcctatgcacaaacccaccctgatcgcgtgcttggcctgattgtgcgcggcatcttcctggcccgcccccaggatatccagtggttctaccaggccggcgcgagccgcctgttcccggactactggcaggactacatcgcgccaatcccggcggaagagcgccacgacatgatcagcgcctaccacaagcgcctgaccggcaatgaccagatcgcccagatgcatgccgccaaggcctggtccacctgggaaggccgcatgctcggcctgtgccccagcccgcagctgatcgagcgcttctccgagccccagcgcgcgttgtcgattgcgcgcatcgagtgccactacttcaccaataactcgttcctggagcccaaccagctgattcgcgatatgcacaagatcgcccatctgccggggatcatcgtgcatggccgctacgatatgatctgcccgctggataatgcctgggagctgcaccaggcctggccgaacagtgagttgcaggtgatccgcgaggcgggccacgcggcgtccgagccgggcatcaccgatgcgctggtgcgtgcggcgggcgatatggcacgacgcctgcttgatctgccccctgaagaagca

SEQ ID NO: 23 - protein, 413 aa, Corynebacterium glutamicum; identical to SEQ ID NO: 17
  UNSURE (359): Other information: Xaa = unknown or other
  UNSURE (401): Other information: Xaa = unknown or other

SEQ ID NO: 24 - DNA, 1239 bp, Corynebacterium glutamicum; identical to SEQ ID NO: 20
  UNSURE (359): Other information: Xaa = unknown or other
  UNSURE (401): Other information: Xaa = unknown or other

SEQ ID NO: 25 - DNA, 27 bp, Artificial Sequence
  Description of Artificial Sequence: Synthetic DNA
ttaaccatggagagaaaaacagtattg

SEQ ID NO: 26 - DNA, 30 bp, Artificial Sequence
  Description of Artificial Sequence: Synthetic DNA
atatggatcctactggcagcacatactttg

SEQ ID NO: 27 - DNA, 21 bp, Artificial Sequence
  Description of Artificial Sequence: Synthetic DNA
caccgcagacggaggatacac

SEQ ID NO: 28 - DNA, 22 bp, Artificial Sequence
  Description of Artificial Sequence: Synthetic DNA
cggacgtcacccaataatcgtg

SEQ ID NO: 29 - DNA, 1428 bp, Bacillus pumilus NRRL B-12025
gtgctttcattgagtaaaaaaactgtacttgtcattgctgacttaggagggtgcccgccccatatgttttatgaaagcgtggcggcatcataccatatcgtttcttatatcccaagaccctttgcgattacaaagggacatgccgagctaatcgaaaaatactccattgccgtcatcaaagaccgtgattattttgagacacacccttcttttgaacaccctgattctatttactgggcacatgatgattatccaaaatcagaagaagaagttgtggaagacttcattcgagtagcttcctttttcaaagcagatgcaatcacgaccaataatgaattattcattgcaccgatggcaaaggccgctgaacgtcttgggctacgaggtgccggtgtcaaggcagccgaaatggcgcgtgataaaagccaaatgagggctgcattcaatgcctctggcgtcaaagcggtgaaaactcagcctgtcacgactttatctgatttccaacaagccattgagtctatcggaacaccgctcattttaaagcctacatatttagccagttctattggcgtcaccttgtttcatgacaaagccggaagtgatgacttgtttttacaagtacaatcgtatttggaaaccataccagtcccagacgctgtcacgtatgaagcaccgtttgtcgctgaaacatatttagagggtgcttacgaagattggtatgaagacgaaggatatgctgattatgtcagtgtagaagggctggtcgtagagggcgaatatctcccttttgtcatacatgataaaacccctcaaatcggctttacagaaacggctcatatcactccgacgatcttagacaatgaagccaagcaaatcatcattgaagcagcaaggaaggcaaatgaagggctaggtcttgaacattgtgcaacccatacagaaatcaaactcatgaaaaatcgagaaactggactgatcgaggcagcggctcgattcgctggctggaatatgatcccgaatattaaaaaagtctttggcgtcgatatggcgaagctattgattgatgtattagttgatggtaaaaaggctgtactgccaaaacagctgctttctggacatacattttatgtagcggactgccacctgtaccctcagcattttaaagagagtgggcttatcccgcctgaagccacacatattaccattgatcatgtgtctattccgcaggaagcattcgttggagatactgcgattgtcagtcaatcattccctgccaaagggactattgtggatcttgaattatttgaagcttttaatggaatcgtatctcttgaattaaaaggatcatcctcacaagatgttgccgcgtccatccgcaacattcagaaacaggcaacgattcagttaatggatgaattagtgaaggga

SEQ ID NO: 30 - DNA, 1416 bp, Bacillus subtilis ATCC 15245 and Bacillus subtilis IAM 1033
atggagagaaaaacagtattggtcatcgctgatcttggaggctgcccgccgcacatgttttataaaagcgctgctgaaaaatataacctggtcagctttattccaagaccttttgcaattacagcctcccatgcagcattgattgaaaaatactcggtcgcggtcataaaagataaagactattttaagagtttagctgattttgagcatcctgattccatttattgggcgcatgaggatcataacaagcctgaggaagaggtcgtcgagcaaatcgtcaaggttgccgaaatgtttggggcggatgccatcacaacaaacaatgaattattcattgctccgatggcgaaagcctgtgaacgtctgggcctgagaggtgccggcgtgcaggcagccgaaaatgccagagataaaaataaaatgagggacgcttttaataaggccggagtcaaatcgatcaaaaacaaacgagtcacaactcttgaagatttccgtgctgctcttgaagagatcggcacacctcttatcttaaagcctacatacttagcgagttcaatcggtgtaacgctgattacggacactgagacggcagaagatgaatttaacagagtcaatgactatctgaaatcaattaacgtgccaaaggcggttacgtttgaagcgccgtttatcgctgaagaatttttacagggtgagtacggagactggtatcaaacagaagggtactccgactatatcagtatagaaggcatcatggctgacggtgagtatttcccgatcgccattcatgataaaacgccgcaaatcgggtttacagagacatcccacattacgccgtccattctggatgaagaggcaaaaaagaaaattgtcgaagctgccaaaaaggcaaatgaagggcttggcctgcaaaattgcgcaacacatacagagatcaagctaatgaaaaacagagaaccgggtttaatagagtcggcagccagattcgcaggctggaatatgattcctaatattaaaaaggtctttggccttgatatggcgcaattattattagatgtcctctgtttcggaaaagacgccgatctgccggacggattattggatcaagagccttattatgttgccgactgccatttgtacccgcagcatttcaaacaaaatggccagattccagaaaccgctgaggatttggtcattgaagcgatcgatattccggacgggcttttaaaaggggatactgaaatcgtttcattttcagccgcagcaccaggcacttcagttgatttgacattgtttgaagctttcaattccattgctgcatttgaactgaaaggcagtaattcacaggatgtggctgaatcaatcagacaaattcagcagcatgcaaagctgacggcaaagtatgtgctgccagta

SEQ ID NO: 31 - DNA, 23 bp, Artificial Sequence
  Description of Artificial Sequence: Synthetic DNA
ccgatggcraaagcstgtraacg

SEQ ID NO: 32 - DNA, 21 bp, Artificial Sequence
  Description of Artificial Sequence: Synthetic DNA
cggcagatcrgcdtcttttcc

SEQ ID NO: 33 - DNA, 26 bp, Artificial Sequence
  Description of Artificial Sequence: Synthetic DNA
gctaggtcttgaacattgtgcaaccc

SEQ ID NO: 34 - DNA, 23 bp, Artificial Sequence
  Description of Artificial Sequence: Synthetic DNA
ggtgttccgatagactcaatggc

SEQ ID NO: 35 - protein, 476 aa, Bacillus pumilus NRRL B-12025
VLSLSKKTVLVIADLGGCPPHMFYESVAASYHIVSYIPRPFAITKGHAELIEKYSIAVIKDRDYFETHPSFEHPDSIYWAHDDYPKSEEEVVEDFIRVASFFKADAITTNNELFIAPMAKAAERLGLRGAGVKAAEMARDKSQMRAAFNASGVKAVKTQPVTTLSDFQQAIESIGTPLILKPTYLASSIGVTLFHDKAGSDDLFLQVQSYLETIPVPDAVTYEAPFVAETYLEGAYEDWYEDEGYADYVSVEGLVVEGEYLPFVIHDKTPQIGFTETAHITPTILDNEAKQIIIEAARKANEGLGLEHCATHTEIKLMKNRETGLIEAAARFAGWNMIPNIKKVFGVDMAKLLIDVLVDGKKAVLPKQLLSGHTFYVADCHLYPQHFKESGLIPPEATHITIDHVSIPQEAFVGDTAIVSQSFPAKGTIVDLELFEAFNGIVSLELKGSSSQDVAASIRNIQKQATIQLMDELVKG

SEQ ID NO: 36 - DNA, 44 bp, Artificial Sequence
  Description of Artificial Sequence: Synthetic DNA
catgccatggagaaaaaaactgtacttgtcattgctgacttagg

SEQ ID NO: 37 - DNA, 38 bp, Artificial Sequence
  Description of Artificial Sequence: Synthetic DNA
cgcggatcccttcactaattcatccattaactgaatcg